SEQUENCE LISTING (2 sequences)

SEQ ID NO: 1 - protein, 21 aa, artificial sequence
  peptomimetic compound
NEDIDQMFSTLLGEMDLLTQS

SEQ ID NO: 2 - protein, 2541 aa, homo sapien
MVALSLKISIGNVVKTMQFEPSTMVYDACRIIRERIPEAPAGPPSDFGLFLSDDDPKKGIWLEAGKALDYYMLRNGDTMEYRKKQRPLKIRMLDGTVKTIMVDDSKTVTDMLMTICARIGITNHDEYSLVRELMEEKKEEITGTLRKDKTLLRDEKKMEKLKQKLHTDDELNWLDHGRTLREQGVEEHETLLLRRKFFYSDQNVDSRDPVQLNLLYVQARDDILNGSHPVSFDKACEFAGFQCQIQFGPHNEQKHKAGFLDLKDFLPKEYVKQKGERKIFQAHKNCGQMSEIEAKVRYVKLARSLKTYGVSFFLVKEKMKGKNKLVPRLLGITKECVMRVDEKTKEVIQEWNLTNIKRWAASPKSFTLDFGDYQDGYYSVQTTEGEQIAQLIAGYIDIILKKKKSKDHFGLEGDEESTMLEDSVSPKKSTVLQQQYNRVGKVEHGSVALPAIMRSGASGPENFQVGSMPPAQQQITSGQMHRGHMPPLTSAQQALTGTINSSMQAVQAAQATLDDFDTLPPLGQDAASKAWRKNKMDESKHEIHSQVDAITAGTASVVNLTAGDPAETDYTAVGCAVTTISSNLTEMSRGVKLLAALLEDEGGSGRPLLQAAKGLAGAVSELLRSAQPASAEPRQNLLQAAGNVGQASGELLQQIGESDTDPHFQDALMQLAKAVASAAAALVLKAKSVAQRTEDSGLQTQVIAAATQCALSTSQLVACTKVVAPTISSPVCQEQLVEAGRLVAKAVEGCVSASQAATEDGQLLRGVGAAATAVTQALNELLQHVKAHATGAGPAGRYDQATDTILTVTENIFSSMGDAGEMVRQARILAQATSDLVNAIKADAEGESDLENSRKLLSAAKILADATAKMVEAAKGAAAHPDSEEQQQRLREAAEGLRMATNAAAQNAIKKKLVQRLEHAAKQAAASATQTIAAAQHAASTPKASAGPQPLLVQSCKAVAEQIPLLVQGVRGSQAQPDSPSAQLALIAASQSFLQPGGKMVAAAKASVPTIQDQASAMQLSQCAKNLGTALAELRTAAQKAQEACGPLEMDSALSVVQNLEKDLQEVKAAARDGKLKPLPGETMEKCTQDLGNSTKAVSSAIAQLLGEVAQGNENYAGIAARDVAGGLRSLAQAARGVAALTSDPAVQAIVLDTASDVLDKASSLIEEAKKAAGHPGDPESQQRLAQVAKAVTQALNRCVSCLPGQRDVDNALRAVGDASKRLLSDSLPPSTGTFQEAQSRLNEAAAGLNQAATELVQASRGTPQDLARASGRFGQDFSTFLEAGVEMAGQAPSQEDRAQVVSNLKGISMSSSKLLLAAKALSTDPAAPNLKSQLAAAARAVTDSINQLITMCTQQAPGQKECDNALRELETVRELLENPVQPINDMSYFGCLDSVMENSKVLGEAMTGISQNAKNGNLPEFGDAISTASKALCGFTEAAAQAAYLVGVSDPNSQAGQQGLVEPTQFARANQAIQMACQSLGEPGCTQAQVLSAATIVAKHTSALCNSCRLASARTTNPTAKRQFVQSAKEVANSTANLVKTIKALDGAFTEENRAQCRAATAPLLEAVDNLSAFASNPEFSSIPAQISPEGRAAMEPIVISAKTMLESAGGLIQTARALAVNPRDPPSWSVLAGHSRTVSDSIKKLITSMRDKAPGQLECETAIAALNSCLRDLDQASLAAVSQQLAPREGISQEALHTQMLTAVQEISHLIEPLANAARAEASQLGHKVSQMAQYFEPLTLAAVGAASKTLSHPQQMALLDQTKTLAESALQLLYTAKEAGGNPKQAAHTQEALEEAVQMMTEAVEDLTTTLNEAASAAGVVGGMVDSITQAINQLDEGPMGEPEGSFVDYQTTMVRTAKAIAVTVQEMVTKSNTSPEELGPLANQLTSDYGRLASEAKPAAVAAENEEIGSHIKHRVQELGHGCAALVTKAGALQCSPSDAYTKKELIECARRVSEKVSHVLAALQAGNRGTQACITAASAVSGIIADLDTTIMFATAGTLNREGTETFADHREGILKTAKVLVEDTKVLVQNAAGSQEKLAQAAQSSVATITRLADVVKLGAASLGAEDPETQVVLINAVKDVAKALGDLISATKAAAGKVGDDPAVWQLKNSAKVMVTNVTSLLKTVKAVEDEATKGTRALEATTEHIRQELAVFCSPEPPAKTSTPEDFIRMTKGITMATAKAVAAGNSCRQEDVIATANLSRRAIADMLRACKEAAYHPEVAPDVRLRALHYGRECANGYLELLDHVLLTLQKPSPELKQQLTGHSKRVAGSVTELIQAAEAMKGTEWVDPEDPTVIAENELLGAAAAIEAAAKKLEQLKPRAKPKEADESLNFEEQILEAAKSIAAATSALVKAASAAQRELVAQGKVGAIPANALDDGQWSQGLISAARMVAAATNNLCEAANAAVQGHASQEKLISSAKQVAASTAQLLVACKVKADQDSEAMKRLQAAGNAVKRASDNLVKAAQKAAAFEEQENETVVVKEKMVGGIAQIIAAQEEMLRKERELEEARKKLAQIRQQQYKFLPSELRDEH

(19) United States
(12) Patent Application Publication
(10) Pub. No.: US 2024/0360186 A1
(43) Pub. Date: Oct. 31, 2024

(54) Treatment Of Integrin-Related Disorders With Stapled Peptides

(71) Applicant: Institute For Cancer Research d/b/a The Research Institute of Fox Chase Cancer Center, Philadelphia, PA (US)

(72) Inventor: Jinhua Wu, Philadelphia, PA (US)

(21) Appl. No.: 18/273,868

(22) PCT Filed: Jan. 28, 2022
(86) PCT No.: PCT/US22/14238

§ 371 Date: Jul. 24, 2023

Related U.S. Application Data
(60) Provisional application No. 63/142,943, filed on Jan. 28, 2021.

(51) Int. Cl. C07K 14/47; A61K 38/00

(52) U.S. Cl. CPC C07K 14/47; A61K 38/00; C07K 2319/10; C07K 2319/30; C07K 2319/60

(57) ABSTRACT

The present disclosure provides, in part, structurally stabilized peptidomimetic compounds that can be used in treatment of integrin-related disorders, to methods of producing structurally stabilized peptidomimetic compounds, and to methods of treating integrin-related disorders by administering structurally stabilized peptidomimetic compounds to subjects in need thereof.

### REFERENCE TO GOVERNMENT GRANTS

This invention was made with government support under Grant No. GM119560 awarded by the National Institute of General Medical Sciences. The government has certain rights in the invention.

### REFERENCE TO SEQUENCE LISTING

This application includes a Sequence Listing filed electronically as a text file named 18530009702SEQ, created on Jan. 26, 2022, with a size of 23 kb. The Sequence Listing is incorporated herein by reference.

### FIELD

The present disclosure is directed, in part, to structurally stabilized peptidomimetic compounds that can be used in treatment of integrin-related disorders, to methods of producing structurally stabilized peptidomimetic compounds, and to methods of treating integrin-related disorders by administering structurally stabilized peptidomimetic compounds to subjects in need thereof.

### BACKGROUND

Integrins are adhesion receptors connecting cells to extracellular matrix ligands and to counter-receptors on other cells. Integrins are obligatory type I αβ heterodimers and molecular machines that undergo large conformational changes in their extracellular domains triggered by signaling molecules inside cells. This process, often referred to as inside-out signaling, is initiated by adaptor molecules that affect the position of the integrin a and R cytoplasmic tails relative to each other and to the plasma membrane. For many, if not all integrins, such conformational changes (“activation”) are required to actuate their adhesive function. Current dogma holds that the ligand binding domain in resting integrins is not readily accessible to adhesive ligands.

Integrin signaling promotes cell proliferation, migration and adhesion, and regulates cell survival via crosstalk with receptor tyrosine kinases. The ectodomains of activated integrin bind the extracellular matrix (ECM) and trigger the outside-in signaling pathway that activates the FAK-Src signaling cascade. Integrins are activated primarily by the cytoskeletal protein talin. Talin also plays an important role in promoting cancer progression and metastasis. In particular, the expression of talin is upregulated in many primary tumors, and its overexpression correlates with metastases of oral squamous cell carcinomas and prostate cancers.

Vertebrates possess two isoforms of talin, talin1 and talin2. Talin1 is ubiquitously expressed in mammalian cells, whereas talin2 is primarily expressed in the heart. Talin1 is also more important in activating integrin in platelets. Activation of integrins by talin is initiated by a Rap1-induced signaling pathway known as the inside-out pathway. Activated RapT recruits an effector protein, Rap1-interacting adaptor molecule (RIAM), which in turn recruits talin to the plasma membrane (PM). Thus, the specific binding to both RapT and talin allows RIAM to play a key role in this pathway by linking cytoplasmic talin to the PM-anchored Rap1. Following its recruitment to the PM by RIAM, talin binds to the intracellular tail of the integrin 3 subunit via the F3 domain from the head region and, in doing so, switches integrin from a low- to a high-affinity state. This activity is suppressed by a competitive intramolecular autoinhibitory interaction between the F3 domain in the head region and the R9 domain in the rod region. Talin also recruits other cytoskeletal proteins, such as actin and vinculin, to stabilize the integrin-mediated focal adhesions.

### SUMMARY

The present disclosure provides peptidomimetic compounds comprising the amino acid sequence NEDIDQMFSTLLGEMDLLTQS (SEQ ID NO: 1) having from 0 to 5 conservative amino acid substitutions, modifications, deletions, or any combination thereof, or a pharmaceutically acceptable salt thereof, wherein the compounds comprise at least one structural stabilization moiety.

The present disclosure also provides methods of treating a subject having an integrin-related disorder, the methods comprising administering a peptidomimetic compound comprising the amino acid sequence NEDIDQMFSTLLGEMDLLTQS (SEQ ID NO: 1) having from 0 to 5 conservative amino acid substitutions, modifications, deletions, or any combination thereof, or a pharmaceutically acceptable salt thereof, wherein the compound comprises at least one structural stabilization moiety.

The present disclosure also provides peptidomimetic compounds comprising the amino acid sequence NEDIDQMFSTLLGEMDLLTQS (SEQ ID NO:1) having from 0 to 5 conservative amino acid substitutions, modifications, deletions, or any combination thereof, or a pharmaceutically acceptable salt thereof, for use in treating a subject having an integrin-related disorder, wherein the compounds comprise at least one structural stabilization moiety.

The present disclosure also provides methods of enhancing the permeability of a plasma membrane, the method comprising contacting the plasma membrane with a peptidomimetic compound comprising the amino acid sequence NEDIDQMFSTLLGEMDLLTQS (SEQ ID NO:1) having from 0 to 5 conservative amino acid substitutions, or a pharmaceutically acceptable salt thereof, wherein the compound comprises at least one structural stabilization moiety.

The present disclosure also provides peptidomimetic compounds comprising the amino acid sequence NEDIDQMFSTLLGEMDLLTQS (SEQ ID NO:1) having from 0 to 5 conservative amino acid substitutions, or a pharmaceutically acceptable salt thereof, for use in enhancing the permeability of a plasma membrane, wherein the compound comprises at least one structural stabilization moiety.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a representative use of a stapled peptide to inhibit integrin function.

FIG. 2 shows the design of a representative talin-binding site (TBS)-derived stapled peptide.

FIG. 3 shows a crystal of talin R7R8 domain in complex with stapled TBS (S-TBS) and diffraction data statistics at 1.8 Å.

FIG. 4 shows the crystal structure of talin R7R8 domain in complex with S-TBS at 1.8-Å (left) and its superposition with the structure of R7R8 domain in complex with the unstapled TBS structure (right).

FIG. 5 shows that the hydrophobic staple enhances membrane permeability.

FIG. 6 shows that S-TBS inhibits integrin activation.

FIG. 7 shows the formula of the representative stapled peptide (Stp(15,19))H2N-NEDIDQMFSTLLGE(S5)DLL(S5)QS-OH.

FIG. 8 shows a crystal structure of S-TBS bound to a talin rod; Panel A shows a structural formula of S-TBS; residue numbers correspond to that in the RIAM sequence; Panel B shows a crystal structure of S-TBS in complex with a talin rod R7R8 domains; talin domains are colored in yellow; S-TBS is colored in cyan, with the C8 linker colored in green; Panel C shows a superposition of the S-TBS bound R8 domain and TBS bound R8 domain (PDB: 4W8P); the TBS:R8 complex is colored in orange (R8) and blue (TBS) (Panel D)); 2Fo-Fc Electron density of the bound S-TBS contoured at 1.2σ is shown in blue mesh (Panel E) shows the C8 hydrocarbon linker and side chains of the residues that form the hydrophobic interface of S-TBS are shown in light gray surface representation.

FIG. 9 shows interactions of S-TBS and talin; Panel A shows determination of dissociation constants of TBS (red line) and S-TBS (blue line) with talin rod R7R8 domains measured by fluorescent polarization (FP) assays; the ordinate represents the concentration of talin in μM and the abscissa represents the polarization values in mP; the disassociation constant K_{d} for TBS was 3.0±0.3 μM and that for S-TBS was 17.1±1.3 μM; Panel B shows determinations of dissociation constants of TBS (red line) and S-TBS (blue line) with talin head domains measured by FP assays; the ordinate represents the concentration of talin in μM and the abscissa represents the polarization values in mP; the K_{d} for TBS was 106±15.5 μM and that for S-TBS was 30.3±4.8 μM; Panel C shows a Western blot from a glutathione S-transferase (GST) pulldown of talin rods (R7R8 domains), talin head (residues 1-430), or β3-THD (integrin β3-talin head domain fusion protein) by GST-TBS or GST-TBS(T14E); Panel D shows that T14E (grey) in TBS is predicted to form a salt bridge interaction with Arg358 in the talin head; Panel E shows that T14E (grey) in TBS is predicted to form a salt bridge interaction with Lys1500 in the talin rod R8 domain.

FIG. 10 shows that TBS and S-TBS enhances membrane permeability; Panel A shows the fluorescence signal of cells treated with 5′ 6-FAM (fluorescein)-labeled TBS (left-hand plot) or fluorescein isothiocyanate (FITC) labeled S-TBS (right-hand plot) at the indicated concentrations; the ordinate represents cell numbers and the abscissa represents the fluorescent index; Panel B shows the permeability indices of TBS (light gray bars) and S-TBS (dark gray bars) measured by flow cytometry; the ordinate represents concentration of TBS or S-TBS, respectively, and the abscissa represents the permeability index; the abbreviation ns indicates no significance while * indicates that p<0.05 and ** indicates that p<0.01; Panel C shows the integrin activity indices of αIIbβ3 in CHO-A5 cells transiently expressing wild-type talin head domain (THD) and treated with TBS or S-TBS; from left to right, the ordinate represents data for dimethyl sulfoxide (DMSO), 100 μM TBS, 10 μM S-TBS, and 100 μM S-TBS while the abscissa represents the integrin activity index; the abbreviation ns indicates no significance while ** indicates that p<0.01; Panel D shows the integrin activity indices of αIIbβ3 in CHO-A5 cells transiently expressing the talin head domain with the W359A mutation (THD-W359A) and treated with TBS or S-TBS; from left to right, the ordinate represents data for dimethyl sulfoxide (DMSO), 100 μM TBS, 10 μM S-TBS, and 100 μM S-TBS while the abscissa represents the integrin activity index; the abbreviation ns refers to no significance.

FIG. 11 shows that TBS and S-TBS exhibit minimal cell toxicity; cell-toxicity assays were conducted in HEK-293 cells (left-hand chart), MCF-7 cells (middle chart), and HeLa cells (right-hand chart); for each chart, the ordinate represents from left to right data for DMSO, 40 μM TBS, 40 μM S-TBS, and 1 μM dasatinib, the bars in orange represents data at 24 hours, and those in gray represents data at 48 hours.

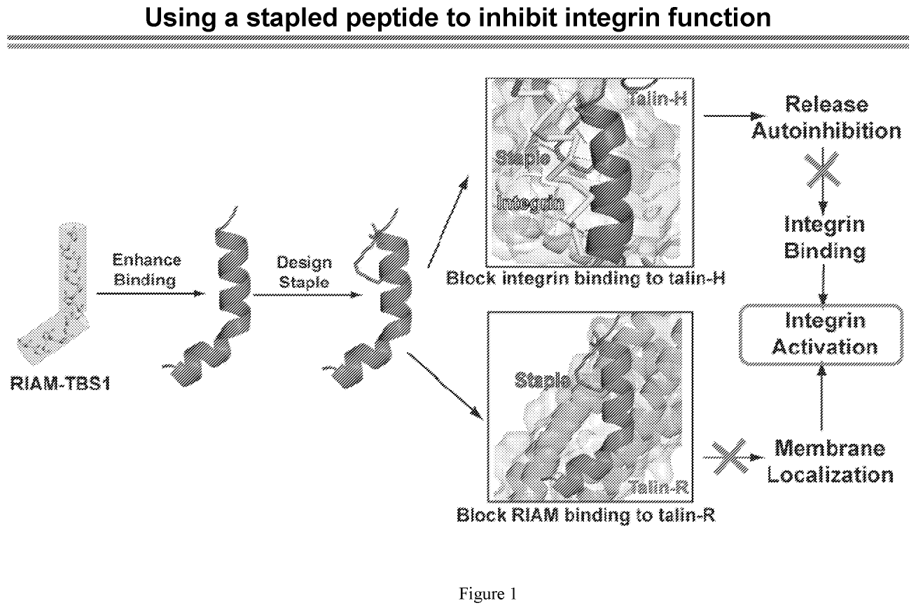

### DESCRIPTION OF EMBODIMENTS

The terminology used herein is for the purpose of describing particular embodiments only and is not intended to be limiting. Various terms relating to aspects of disclosure are used throughout the specification and claims. Such terms are to be given their ordinary meaning in the art, unless otherwise indicated. Other specifically defined terms are to be construed in a manner consistent with the definition provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Various terms relating to embodiments of the present disclosure are used throughout the specification and claims. Such terms are to be given their ordinary meaning in the art, unless otherwise indicated. Other specifically defined terms are to be construed in a manner consistent with the definition provided in this document.

As used herein, the singular forms “a,” “an,” and “the” include plural referents unless expressly stated otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by +10% and remain within the scope of the disclosed embodiments.

As used herein, the terms “administer” or “administering” means to introduce a compound or composition to a subject. The term is not limited to any specific mode of delivery. Furthermore, depending on the mode of delivery, the administering can be carried out by various individuals, including, for example, a health-care professional (e.g., physician, nurse, etc.), a pharmacist, or the subject (i.e., self-administration).

As used herein, the term “aliphatic” or “aliphatic group” refers to a saturated or unsaturated, linear or branched, cyclic (non-aromatic) or heterocyclic (non-aromatic), hydrocarbon or hydrocarbon group and encompasses alkyl, alkenyl, and alkynyl groups, and alkanes, alkene, and alkynes, for example.

As used herein, the term “alkane” refers to a saturated aliphatic hydrocarbon which can be straight or branched, having 8 to 12, 9 to 12, 10 to 12, 11 to 12, 8 to 11, 8 to 10, 8 to 9, 9 to 11, 9 to 10, or 10 to 11 carbon atoms, where the stated range of carbon atoms includes each intervening integer individually, as well as sub-ranges. Examples of alkane include, but are not limited to methane, ethane, propane, butane, pentane, and the like. Reference to “alkane” includes unsubstituted and substituted forms of the hydrocarbon.

As used herein, the term “alkene” refers to an aliphatic hydrocarbon which can be straight or branched, containing at least one carbon-carbon double bond, having 8 to 12, 9 to 12, 10 to 12, 11 to 12, 8 to 11, 8 to 10, 8 to 9, 9 to 11, 9 to 10, or 10 to 11 carbon atoms, where the stated range of carbon atoms includes each intervening integer individually, as well as sub-ranges. Examples of alkene groups include, but are not limited to, ethene, propene, and the like. Reference to “alkene” includes unsubstituted and substituted forms of the hydrocarbon.

As used herein, the term “alkyne” refers to straight or branched chain hydrocarbon groups having 8 to 12, 9 to 12, 10 to 12, 11 to 12, 8 to 11, 8 to 10, 8 to 9, 9 to 11, 9 to 10, or 10 to 11 carbon atoms and at least one triple carbon to carbon bond. Reference to “alkyne” includes unsubstituted and substituted forms of the hydrocarbon.

As used herein, the terms “binds” and “binding” or grammatical equivalents refer to compounds having affinity for each other.

As used herein, the term “carrier” means a diluent, adjuvant, or excipient with which a compound is administered in a composition.

As used herein, the term, “compound” means all stereoisomers, tautomers, isotopes, and polymorphs of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive and open-ended and include the options following the terms, and do not exclude additional, unrecited elements or method steps.

As used herein, the phrase “conservative amino acid substitution” means one in which an amino acid residue is substituted by another amino acid residue having a side chain (R group) with similar chemical properties (e.g., charge and/or hydrophobicity). In general, a conservative amino acid substitution will not substantially change the functional properties of a polypeptide.

As used herein, the term “hydrocarbon” refers to molecules or segments of molecules containing primarily hydrogen and carbon atoms. As used herein, the term “Cₙ” hydrocarbon wherein n is a positive integer, e.g., 1, 2, 3, 4, etc., means a hydrocarbon having n number of carbon atom(s) per molecule. The term “Cₙ₊” hydrocarbon wherein n is a positive integer, e.g., 1, 2, 3, 4, etc., as used herein, means a hydrocarbon having at least n number of carbon atom(s) per molecule. The term “Cₙ₋” hydrocarbon wherein n is a positive integer, e.g., 1, 2, 3, 4, etc., used herein, means a hydrocarbon having no more than n number of carbon atom(s) per molecule.

As used herein, the phrase “in need thereof” means that the “individual,” “subject,” or “patient” has been identified as having a need for the particular method, prevention, or treatment. In some embodiments, the identification can be by any means of diagnosis. In any of the methods, preventions, and treatments described herein, the “individual,” “subject,” or “patient” can be in need thereof.

As used herein, “peptidomimetic” refers to a mimetic of a peptide which includes an alteration of the normal peptide chemistry. Peptidomimetics typically enhance a property of the original peptide, such as increased stability, increased efficacy, enhanced delivery, increased half-life, etc. Methods of making peptidomimetics based upon a known polypeptide sequence is described, for example, in U.S. Pat. Nos. 5,631,280, 5,612,895, and 5,579,250. Use of peptidomimetics can involve the incorporation of a non-amino acid residue with non-amide linkages at a particular position. Suitable examples of unnatural amino acids include, but are not limited to, β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-omithine, Boc-p-nitro-L-phenylalanine, Boc-hydroxyproline, and Boc-L-thioproline.

As used herein, the phrase “pharmaceutically acceptable” means that the compounds, materials, compositions, and/or dosage forms are within the scope of sound medical judgment and are suitable for use in contact with tissues of humans and other animals. In some embodiments, “pharmaceutically acceptable” means approved by a regulatory agency of the Federal government or a state government or listed in the U.S. Pharmacopeia or other generally recognized pharmacopeia for use in animals, and more particularly in humans. In some embodiments, the pharmaceutically acceptable compounds, materials, compositions, and/or dosage forms result in no persistent detrimental effect on the subject, or on the general health of the subject being treated. However, it will be recognized that transient effects, such as minor irritation or a “stinging” sensation, are common with administration of medicament and the existence of such transient effects is not inconsistent with the composition, formulation, or ingredient (e.g., excipient) in question.

As used herein, the term “pharmaceutical composition” means a composition that may be administered to a mammalian host containing conventional non-toxic carriers, diluents, adjuvants, vehicles and the like.

As used herein, the term “subject” means any animal, such as a mammal. A mammalian subject may be a farm animal (e.g., sheep, horse, cow, pig), a companion animal (e.g., cat, dog), a rodent or laboratory animal (e.g., mouse, rat, rabbit), or a non-human primate (e.g., old world monkey, new world monkey). In some embodiments, the mammal is a human. In some embodiments, the subject is a patient in the care of a physician.

As used herein, the term “substituted” refers to any one or more hydrogen atoms on the designated atom (e.g., a carbon atom) that can be replaced with a selection from the indicated group (e.g., halide, hydroxyl, alkyl, and the like), provided that the designated atom's normal valence is not exceeded.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

As used herein, the term “unit dose” is a discrete amount of a pharmaceutical composition comprising a predetermined amount of the active ingredient.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The present disclosure provides peptidomimetic compounds, or pharmaceutically acceptable salts thereof. In some embodiments, the peptidomimetic compounds interact directly with TLN1. In some embodiments, the peptidomimetic compounds stabilize the autoinhibitory conformation of TLN1. In some embodiments, the peptidomimetic compounds stabilize the autoinhibitory conformation of TLN1 through interaction with the TLN1 head domain. In some embodiments, the peptidomimetic compounds stabilize the autoinhibitory conformation of TLN1 through interaction with the TLN1 ROD domain. In some embodiments, the peptidomimetic compounds stabilize the autoinhibitory conformation of TLN1 through interaction with the R07-R09 segment of the ROD domain, wherein the R07-R09 segment comprises or consists of amino acid positions corresponding to positions 1357 to 1848 according to SEQ ID NO:2. In some embodiments, the peptidomimetic compounds stabilize the autoinhibitory conformation of TLN1 through interaction with the R08 segment of the ROD domain, wherein the R08 segment comprises or consists of amino acid positions corresponding to positions 1487 to 1548 according to SEQ ID NO:2.

In some embodiments, the peptidomimetic compounds, or pharmaceutically acceptable salts thereof, comprise the amino acid sequence NEDIDQMFSTLLGEMDLLTQS (SEQ ID NO:1). In some embodiments, the peptidomimetic compounds have from 0 to 5 conservative amino acid substitutions, modifications, deletions, or any combination thereof. In some embodiments, the peptidomimetic compounds have from 0 to 4 conservative amino acid substitutions, modifications, deletions, or any combination thereof. In some embodiments, the peptidomimetic compounds have from 0 to 3 conservative amino acid substitutions, modifications, deletions, or any combination thereof. In some embodiments, the peptidomimetic compounds have from 0 to 2 conservative amino acid substitutions, modifications, deletions, or any combination thereof. In some embodiments, the peptidomimetic compounds have 0 or 1 conservative amino acid substitution, modification, or deletion. In some embodiments, the peptidomimetic compounds have from 1 to 5 conservative amino acid substitutions, modifications, deletions, or any combination thereof. In some embodiments, the peptidomimetic compounds have from 2 to 5 conservative amino acid substitutions, modifications, deletions, or any combination thereof. In some embodiments, the peptidomimetic compounds have from 3 to 5 conservative amino acid substitutions, modifications, deletions, or any combination thereof. In some embodiments, the peptidomimetic compounds have from 4 to 5 conservative amino acid substitutions, modifications, deletions, or any combination thereof. In some embodiments, the peptidomimetic compounds have 0 conservative amino acid substitutions, modifications, or deletions. In some embodiments, the peptidomimetic compounds have 1 conservative amino acid substitution, modification, or deletion. In some embodiments, the peptidomimetic compounds have 2 conservative amino acid substitutions, modifications, deletions, or any combination thereof. In some embodiments, the peptidomimetic compounds have 3 conservative amino acid substitutions, modifications, deletions, or any combination thereof. In some embodiments, the peptidomimetic compounds have 4 conservative amino acid substitutions, modifications, deletions, or any combination thereof. In some embodiments, the peptidomimetic compounds have 5 conservative amino acid substitutions, modifications, deletions, or any combination thereof. In some embodiments, the peptidomimetic compounds comprise at least one structural stabilization moiety.

In some embodiments, the peptidomimetic compounds, or pharmaceutically acceptable salts thereof, comprise the amino acid sequence NEDIDQMFSTLLGEMDLLTQS (SEQ ID NO:1). In some embodiments, the peptidomimetic compounds have from 0 to 5 conservative amino acid substitutions. In some embodiments, the peptidomimetic compounds have from 0 to 4 conservative amino acid substitutions. In some embodiments, the peptidomimetic compounds have from 0 to 3 conservative amino acid substitutions. In some embodiments, the peptidomimetic compounds have from 0 to 2 conservative amino acid substitutions. In some embodiments, the peptidomimetic compounds have 0 or 1 conservative amino acid substitution. In some embodiments, the peptidomimetic compounds have from 1 to 5 conservative amino acid substitutions. In some embodiments, the peptidomimetic compounds have from 2 to 5 conservative amino acid substitutions. In some embodiments, the peptidomimetic compounds have from 3 to 5 conservative amino acid substitutions. In some embodiments, the peptidomimetic compounds have from 4 to 5 conservative amino acid substitutions. In some embodiments, the peptidomimetic compounds have 0 conservative amino acid substitutions. In some embodiments, the peptidomimetic compounds have 1 conservative amino acid substitution. In some embodiments, the peptidomimetic compounds have 2 conservative amino acid substitutions. In some embodiments, the peptidomimetic compounds have 3 conservative amino acid substitutions. In some embodiments, the peptidomimetic compounds have 4 conservative amino acid substitutions. In some embodiments, the peptidomimetic compounds have 5 conservative amino acid substitutions. In some embodiments, the peptidomimetic compounds comprise at least one structural stabilization moiety.

In some embodiments, the peptidomimetic compounds comprise an amino acid sequence that is at least 70% identical to SEQ ID NO:1. In some embodiments, the peptidomimetic compounds comprise an amino acid sequence that is at least 75% identical to SEQ ID NO:1. In some embodiments, the peptidomimetic compounds comprise an amino acid sequence that is at least 80% identical to SEQ ID NO:1. In some embodiments, the peptidomimetic compounds comprise an amino acid sequence that is at least 85% identical to SEQ ID NO:1. In some embodiments, the peptidomimetic compounds comprise an amino acid sequence that is at least 90% identical to SEQ ID NO:1. In some embodiments, the peptidomimetic compounds comprise an amino acid sequence that is at least 95% identical to SEQ ID NO:1. In some embodiments, the peptidomimetic compounds comprise an amino acid sequence that is at least 96% identical to SEQ ID NO:1. In some embodiments, the peptidomimetic compounds comprise an amino acid sequence that is at least 97% identical to SEQ ID NO:1. In some embodiments, the peptidomimetic compounds comprise an amino acid sequence that is at least 98% identical to SEQ ID NO:1. In some embodiments, the peptidomimetic compounds comprise an amino acid sequence that is at least 99% identical to SEQ ID NO:1. In some embodiments, the peptidomimetic compounds comprise an amino acid sequence that is at least 100% identical to SEQ ID NO: 1. In some embodiments, the peptidomimetic compounds consist of the amino acid sequence set forth in SEQ ID NO:1.

In some embodiments, the peptidomimetic compounds comprise a chemically modified peptide having the amino acid sequence NEDIDQMFSTLLGEMDLLTQS (SEQ ID NO: 1). Numerous peptide modifications are known in the art and are described, for example, in U.S. Pat. No. 9,593,154 and PCT Publication No. WO 2018/175589. Suitable modifications include, but are not limited to, substitutions of natural and unnatural amino acids, or amino acid analogs, as well as modifications within or between various amino acids. When referring to the location of particular amino acids or analogs within SEQ ID NO:1 or SEQ ID NO:1 analogs, those locations can be referred to as “positions” within the peptide, with the positions numbered from 1 (Asn in SEQ ID NO: 1) to 21 (Ser in SEQ ID NO:1). For example, the Phe residue occupies “position 8.” When referring to particular amino acids within SEQ ID NO:1 or SEQ ID NO:1 analogs, those amino acids are sometimes referred to by a single letter code designations followed by the position within SEQ ID NO:1. For example, the Phe residue at position 8 can be referred to as “F8”.

In some embodiments, the at least one structural stabilization moiety links two nonadjacent amino acid residues within SEQ ID NO:1. In some embodiments, the two nonadjacent amino acid residues within SEQ ID NO:1 are 2 to 7 amino acids apart. In some embodiments, the two nonadjacent amino acid residues within SEQ ID NO:1 are 2 to 6 amino acids apart. In some embodiments, the two nonadjacent amino acid residues within SEQ ID NO:1 are 2 to 5 amino acids apart. In some embodiments, the two nonadjacent amino acid residues within SEQ ID NO:1 are 2 to 4 amino acids apart. In some embodiments, the two nonadjacent amino acid residues within SEQ ID NO:1 are 2 or 3 amino acids apart. In some embodiments, the two nonadjacent amino acid residues within SEQ ID NO:1 are 3 to 7 amino acids apart. In some embodiments, the two nonadjacent amino acid residues within SEQ ID NO:1 are 4 to 7 amino acids apart. In some embodiments, the two nonadjacent amino acid residues within SEQ ID NO:1 are 5 to 7 amino acids apart. In some embodiments, the two nonadjacent amino acid residues within SEQ ID NO:1 are 6 or 7 amino acids apart. In some embodiments, the two nonadjacent amino acid residues within SEQ ID NO:1 are 2 amino acids apart. In some embodiments, the two nonadjacent amino acid residues within SEQ ID NO:1 are 3 amino acids apart. In some embodiments, the two nonadjacent amino acid residues within SEQ ID NO:1 are 4 amino acids apart. In some embodiments, the two nonadjacent amino acid residues within SEQ ID NO:1 are 5 amino acids apart. In some embodiments, the two nonadjacent amino acid residues within SEQ ID NO:1 are 6 amino acids apart. In some embodiments, the two nonadjacent amino acid residues within SEQ ID NO:1 are 7 amino acids apart.

In some embodiments, the at least one structural stabilization moiety links any two non-adjacent amino acids within positions 1 to 9 of SEQ ID NO:1. In some embodiments, the peptidomimetic compounds comprise a structural stabilization moiety linking amino acids N1 and D3 of SEQ ID NO:1. In some embodiments, the peptidomimetic compounds comprise a structural stabilization moiety linking amino acids N1 and D5 of SEQ ID NO:1. In some embodiments, the peptidomimetic compounds comprise a structural stabilization moiety linking amino acids N1 and Q6 of SEQ ID NO:1. In some embodiments, the peptidomimetic compounds comprise a structural stabilization moiety linking amino acids N1 and M7 of SEQ ID NO:1. In some embodiments, the peptidomimetic compounds comprise a structural stabilization moiety linking amino acids E2 and D5 of SEQ ID NO:1. In some embodiments, the peptidomimetic compounds comprise a structural stabilization moiety linking amino acids E2 and Q6 of SEQ ID NO:1. In some embodiments, the peptidomimetic compounds comprise a structural stabilization moiety linking amino acids E2 and M7 of SEQ ID NO:1. In some embodiments, the peptidomimetic compounds comprise a structural stabilization moiety linking amino acids E2 and S9 of SEQ ID NO:1. In some embodiments, the peptidomimetic compounds comprise a structural stabilization moiety linking amino acids D3 and D5 of SEQ ID NO:1. In some embodiments, the peptidomimetic compounds comprise a structural stabilization moiety linking amino acids D3 and Q6 of SEQ ID NO:1. In some embodiments, the peptidomimetic compounds comprise a structural stabilization moiety linking amino acids D3 and M7 of SEQ ID NO:1. In some embodiments, the peptidomimetic compounds comprise a structural stabilization moiety linking amino acids D3 and S9 of SEQ ID NO:1. In some embodiments, the peptidomimetic compounds comprise a structural stabilization moiety linking amino acids D5 and M7 of SEQ ID NO:1. In some embodiments, the peptidomimetic compounds comprise a structural stabilization moiety linking amino acids D5 and S9 of SEQ ID NO:1.

In some embodiments, the at least one structural stabilization moiety links any two non-adjacent amino acids within positions 10 to 21 of SEQ ID NO:1. In some embodiments, the peptidomimetic compounds comprise a structural stabilization moiety linking amino acids T10 and L12 of SEQ ID NO:1. In some embodiments, the peptidomimetic compounds comprise a structural stabilization moiety linking amino acids T10 and G13 of SEQ ID NO:1. In some embodiments, the peptidomimetic compounds comprise a structural stabilization moiety linking amino acids T10 and E14 of SEQ ID NO:1. In some embodiments, the peptidomimetic compounds comprise a structural stabilization moiety linking amino acids T10 and M15 of SEQ ID NO:1. In some embodiments, the peptidomimetic compounds comprise a structural stabilization moiety linking amino acids T10 and D16 of SEQ ID NO:1. In some embodiments, the peptidomimetic compounds comprise a structural stabilization moiety linking amino acids T10 and L17 of SEQ ID NO:1. In some embodiments, the peptidomimetic compounds comprise a structural stabilization moiety linking amino acids L12 and E14 of SEQ ID NO:1. In some embodiments, the peptidomimetic compounds comprise a structural stabilization moiety linking amino acids L12 and M15 of SEQ ID NO: 1. In some embodiments, the peptidomimetic compounds comprise a structural stabilization moiety linking amino acids L12 and D16 of SEQ ID NO:1. In some embodiments, the peptidomimetic compounds comprise a structural stabilization moiety linking amino acids L12 and L17 of SEQ ID NO:1. In some embodiments, the peptidomimetic compounds comprise a structural stabilization moiety linking amino acids L12 and T19 of SEQ ID NO:1. In some embodiments, the peptidomimetic compounds comprise a structural stabilization moiety linking amino acids L12 and Q20 of SEQ ID NO:1. In some embodiments, the peptidomimetic compounds comprise a structural stabilization moiety linking amino acids G13 and M15 of SEQ ID NO:1. In some embodiments, the peptidomimetic compounds comprise a structural stabilization moiety linking amino acids G13 and D16 of SEQ ID NO:1. In some embodiments, the peptidomimetic compounds comprise a structural stabilization moiety linking amino acids G13 and L17 of SEQ ID NO: 1. In some embodiments, the peptidomimetic compounds comprise a structural stabilization moiety linking amino acids G13 and T19 of SEQ ID NO:1. In some embodiments, the peptidomimetic compounds comprise a structural stabilization moiety linking amino acids G13 and Q20 of SEQ ID NO:1. In some embodiments, the peptidomimetic compounds comprise a structural stabilization moiety linking amino acids G13 and S21 of SEQ ID NO:1. In some embodiments, the peptidomimetic compounds comprise a structural stabilization moiety linking amino acids E14 and D16 of SEQ ID NO:1. In some embodiments, the peptidomimetic compounds comprise a structural stabilization moiety linking amino acids E14 and L17 of SEQ ID NO:1. In some embodiments, the peptidomimetic compounds comprise a structural stabilization moiety linking amino acids E14 and T19 of SEQ ID NO:1. In some embodiments, the peptidomimetic compounds comprise a structural stabilization moiety linking amino acids E14 and Q20 of SEQ ID NO:1. In some embodiments, the peptidomimetic compounds comprise a structural stabilization moiety linking amino acids E14 and S21 of SEQ ID NO:1. In some embodiments, the peptidomimetic compounds comprise a structural stabilization moiety linking amino acids M15 and L17 of SEQ ID NO:1. In some embodiments, the peptidomimetic compounds comprise a structural stabilization moiety linking amino acids M15 and T19 of SEQ ID NO:1. In some embodiments, the peptidomimetic compounds comprise a structural stabilization moiety linking amino acids M15 and Q20 of SEQ ID NO:1. In some embodiments, the peptidomimetic compounds comprise a structural stabilization moiety linking amino acids M15 and S21 of SEQ ID NO:1. In some embodiments, the peptidomimetic compounds comprise a structural stabilization moiety linking amino acids D16 and T19 of SEQ ID NO:1. In some embodiments, the peptidomimetic compounds comprise a structural stabilization moiety linking amino acids D16 and Q20 of SEQ ID NO:1. In some embodiments, the peptidomimetic compounds comprise a structural stabilization moiety linking amino acids D16 and S21 of SEQ ID NO:1. In some embodiments, the peptidomimetic compounds comprise a structural stabilization moiety linking amino acids L17 and T19 of SEQ ID NO:1. In some embodiments, the peptidomimetic compounds comprise a structural stabilization moiety linking amino acids L17 and Q20 of SEQ ID NO:1. In some embodiments, the peptidomimetic compounds comprise a structural stabilization moiety linking amino acids L17 and S21 of SEQ ID NO:1. In some embodiments, the peptidomimetic compounds comprise a structural stabilization moiety linking amino acids T19 and S21 of SEQ ID NO:1.

In some embodiments, the at least one structural stabilization moiety links any amino acid except 14, F8, L11, and L18 to another non-adjacent amino acid within SEQ ID NO:1. In some embodiments, the at least one structural stabilization moiety links the amino acid at position 15 to the amino acid at position 19 of SEQ ID NO:1.

In some embodiments, the peptidomimetic compounds are modified with a structural stabilization moiety that functions to stabilize the structure of the compound. Protein structures, such as alpha helices, when removed from the context within a protein have a propensity for unraveling and forming random coils, which are, in most cases, biologically less active, or even inactive, and are highly susceptible to proteolytic degradation. Methods of stabilizing peptide structures include introduction of an (intramolecular) cross-link (or staple) linking two non-adjacent amino acids. Stabilized peptides as described herein include stapled peptides and stitched peptides as well as peptides containing multiple stitches, multiple staples or a mix or staples and stitches, or other chemical strategies for structural reinforcement (see, for example, Balaram, Cur. Opin. Struct. Biol., 1992, 2, 845; Kemp et al., J. Am. Chem. Soc., 1996, 118, 4240; Omer et al., J. Am. Chem. Soc., 2001, 123, 5382; Chin et al., Int. Ed., 2001, 40, 3806; Chapman et al., J. Am. Chem. Soc., 2004, 126, 12252; Home et al., Chem. Int. Ed., 2008, 47, 2853; Madden et al., Chem. Commun. (Camb), 2009, 37, 5588-5590; Lau et al., Chem. Soc. Rev., 2015, 44, 91-102; and Gunnoo et al., Org. Biomol. Chem., 2016, 14, 8002-8013).

In some embodiments, the peptidomimetic compounds described herein can be stabilized by peptide stapling (see, for example, Walensky, J. Med. Chem., 2014, 57, 6275-6288). A peptide is stabilized in that it maintains its native secondary structure. For example, stapling allows a polypeptide, predisposed to have an alpha-helical secondary structure, to maintain its native alpha-helical conformation. This secondary structure increases resistance of the peptidomimetic compounds to proteolytic cleavage and heat, and also may increase target binding affinity, hydrophobicity, and cell permeability. Accordingly, the stapled (cross-linked) peptidomimetic compounds described herein have improved biological activity relative to a corresponding non-stapled (un-cross-linked) peptidomimetic compounds.

Peptide stapling can occur where two olefin containing side-chains (e.g., cross-linkable side chains) present in a polypeptide chain are covalently joined (e.g., “stapled together”) using a ring-closing metathesis (RCM) reaction to form a cross-linked ring (see, for example, Blackwell et al., J. Org. Chem., 2001, 66, 5291-5302; Angew et al., Chem. Int. Ed., 1994, 37, 3281). Peptide stapling includes the joining of two (e.g., at least one pair of) double bond-containing side-chains, triple bond-containing side-chains, or double bond-containing and triple bond-containing side chain, which may be present in a polypeptide chain, using any number of reaction conditions and/or catalysts to facilitate such a reaction, to provide a singly “stapled” polypeptide. Multiply stapled polypeptides include those polypeptides containing more than one individual staple, and may contain two, three, or more independent staples of various spacing. Additionally, peptide stitching can include multiple and tandem “stapling” events in a single polypeptide chain to provide a “stitched” (e.g., tandem or multiply stapled) polypeptide, in which two staples, for example, are linked to a common residue. Peptide stitching is disclosed, for example, in PCT Publication No. WO 2008/121767 and WO 2010/068684. In some instances, staples can retain the unsaturated bond or can be reduced.

In some embodiments, one or more of the peptidomimetic compounds described herein can be stabilized by hydrocarbon stapling. In some embodiments, the stapled peptidomimetic compounds comprise or consist of SEQ ID NO:1, and comprise at least two (e.g., 2, 3, 4, 5, or 6) amino acid substitutions, modifications, deletions, or any combination thereof, wherein the substituted amino acids are non-adjacent. In some embodiments, the substituted amino acids are separated by one, two, three, four, five, six, or seven amino acids, and wherein the substituted amino acids are non-natural amino acids with olefinic side chains. Examples of unnatural amino acids include, but are not limited to, 4-hydroxyproline, desmosine, gamma-aminobutyric acid, beta cyanoalanine, norvaline, 4-(E)-butenyl-4(R)-methyl-N-methyl-L-threonine, N-methyl-L leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino cyclohexanecarboxylic acid, 4-piperidylacetic acid, 4-amino-methylpyrrole-2-carboxylic acid, 2,4-diaminobutyric acid, 2,3-diaminopropionic acid, 2,4-diaminobutyric acid, 2 aminoheptanedioic acid, 4-(aminomethyl)benzoic acid, 4-aminobenzoic acid, ortho-, meta and para-substituted phenylalanines (e.g., substituted with —C(═O)C₆H₅, —CF₃, —CN, -halo, NO₂, and/or CH₃), disubstituted phenylalanines, substituted tyrosines (e.g., further substituted with C═O)C₆H, —CF₃, —CN; -halo; —NO₂, and/or CH₃), and statine. Additionally, amino acids can be derivatized to include amino acid residues that are hydroxylated, phosphorylated, sulfonated, acylated, or glycosylated.

Hydrocarbon stapled peptidomimetic compounds can include one or more tethers (linkages) between two non-natural amino acids, wherein the tether significantly enhances the u-helical secondary structure of the polypeptide. In some embodiments, the tether can extend across the length of one or two helical turns (i.e., about 3, 4, or 7 amino acids). Accordingly, amino acids positioned at i and i+3, i and i+4, or i and i+7 are ideal candidates for chemical modification and cross-linking. Thus, for example, where a peptide has the sequence . . . X1, X2, X3, X4, X5, X6, X7, X8, X9 . . . , cross-links between X1 and X4, or between X1 and X5, or between X1 and X8 are useful hydrocarbon stapled forms of that peptide, as are cross-links between X2 and X5, or between X2 and X6, or between X2 and X9, etc. The use of multiple cross-links (e.g., 2, 3, 4, or more) is also contemplated. The use of multiple cross-links is very effective at stabilizing and optimizing the peptide, especially with increasing peptide length. Thus, the present disclosure encompasses the incorporation of more than one cross-link within the polypeptide sequence to either further stabilize the sequence or facilitate the structural stabilization, proteolytic resistance, acid stability, thermal stability, cellular permeability, and/or biological activity enhancement of longer polypeptide stretches. Additional description regarding making and use of hydrocarbon stapled polypeptides can be found, e.g., in U.S. Patent Publication Nos. 2012/0172285, 2010/0286057, and 2005/0250680.

In some embodiments, when a staple is at the i and i+3 residues, R-propenylalanine and S-pentenylalanine (e.g., S-2-(4′-pentenyl) alanine), or R-pentenylalanine and S-pentenylalanine are substituted for the amino acids at those positions. In some embodiments, when a staple is at the i and i+4 residues, S-pentenyl alanine is substituted for the amino acids at those positions. In some embodiments, when a staple is at the i and i+7 residues, S-pentenyl alanine and R-octenyl alanine are substituted for the amino acids at those positions. In some embodiments, when the peptide is stitched, the amino acids of the peptide to be involved in the “stitch” are substituted with bis-pentenylglycine, S-pentenylalanine, and R-octenylalanine, or bis-pentenylglycine, S-octenylalanine, and R-octenylalanine.

In some embodiments, the structural stabilization moiety is a hydrocarbon having from about 5 to about 20 carbons. In some embodiments, the hydrocarbon comprises a substituted or unsubstituted alkane comprising from about 5 to about 20 carbons, a substituted or unsubstituted alkene comprising from about 5 to about 20 carbons, or a substituted or unsubstituted alkyne comprising from about 5 to about 20 carbons. In some embodiments, the hydrocarbon comprises a substituted or unsubstituted alkane comprising from about 5 to about 20 carbons. In some embodiments, the hydrocarbon comprises a substituted or unsubstituted alkene comprising from about 5 to about 20 carbons. In some embodiments, the hydrocarbon comprises a substituted or unsubstituted alkyne comprising from about 5 to about 20 carbons.

In some embodiments, the structural stabilization moiety is a hydrocarbon having from about 8 to about 12 carbons. In some embodiments, the hydrocarbon comprises a substituted or unsubstituted alkane comprising from about 8 to about 12 carbons, a substituted or unsubstituted alkene comprising from about 8 to about 12 carbons, or a substituted or unsubstituted alkyne comprising from about 8 to about 12 carbons. In some embodiments, the hydrocarbon comprises a substituted or unsubstituted alkane comprising from about 8 to about 12 carbons. In some embodiments, the hydrocarbon comprises a substituted or unsubstituted alkene comprising from about 8 to about 12 carbons. In some embodiments, the hydrocarbon comprises a substituted or unsubstituted alkyne comprising from about 8 to about 12 carbons.

In some embodiments, the hydrocarbon comprises a substituted or unsubstituted alkane comprising about 8 carbons, a substituted or unsubstituted alkene comprising about 8 carbons, or a substituted or unsubstituted alkyne comprising about 8 carbons. In some embodiments, the hydrocarbon comprises a substituted or unsubstituted alkane comprising about 8 carbons. In some embodiments, the hydrocarbon comprises a substituted or unsubstituted alkene comprising about 8 carbons. In some embodiments, the hydrocarbon comprises a substituted or unsubstituted alkyne comprising about 8 carbons.

In some embodiments, the hydrocarbon comprises a substituted or unsubstituted alkane comprising about 9 carbons, a substituted or unsubstituted alkene comprising about 9 carbons, or a substituted or unsubstituted alkyne comprising about 9 carbons. In some embodiments, the hydrocarbon comprises a substituted or unsubstituted alkane comprising about 9 carbons. In some embodiments, the hydrocarbon comprises a substituted or unsubstituted alkene comprising about 9 carbons. In some embodiments, the hydrocarbon comprises a substituted or unsubstituted alkyne comprising about 9 carbons.

In some embodiments, the hydrocarbon comprises a substituted or unsubstituted alkane comprising about 10 carbons, a substituted or unsubstituted alkene comprising about 10 carbons, or a substituted or unsubstituted alkyne comprising about 10 carbons. In some embodiments, the hydrocarbon comprises a substituted or unsubstituted alkane comprising about 10 carbons. In some embodiments, the hydrocarbon comprises a substituted or unsubstituted alkene comprising about 10 carbons. In some embodiments, the hydrocarbon comprises a substituted or unsubstituted alkyne comprising about 10 carbons.

In some embodiments, the hydrocarbon comprises a substituted or unsubstituted alkane comprising about 11 carbons, a substituted or unsubstituted alkene comprising about 11 carbons, or a substituted or unsubstituted alkyne comprising about 11 carbons. In some embodiments, the hydrocarbon comprises a substituted or unsubstituted alkane comprising about 11 carbons. In some embodiments, the hydrocarbon comprises a substituted or unsubstituted alkene comprising about 11 carbons. In some embodiments, the hydrocarbon comprises a substituted or unsubstituted alkyne comprising about 11 carbons.

In some embodiments, the hydrocarbon comprises a substituted or unsubstituted alkane comprising about 12 carbons, a substituted or unsubstituted alkene comprising about 12 carbons, or a substituted or unsubstituted alkyne comprising about 12 carbons. In some embodiments, the hydrocarbon comprises a substituted or unsubstituted alkane comprising about 12 carbons. In some embodiments, the hydrocarbon comprises a substituted or unsubstituted alkene comprising about 12 carbons. In some embodiments, the hydrocarbon comprises a substituted or unsubstituted alkyne comprising about 12 carbons.

While hydrocarbon structural stabilization moieties are common, other structural stabilization moieties can also be employed in the peptidomimetic compounds described herein. For example, the structural stabilization moieties can include one or more of an ether, thioether, ester, amine, or amide, or triazole moiety. In some embodiments, a naturally occurring amino acid side chain can be incorporated into a structural stabilization moiety. For example, a structural stabilization moiety can be coupled with a functional group such as the hydroxyl in serine, the thiol in cysteine, the primary amine in lysine, the acid in aspartate or glutamate, or the amide in asparagine or glutamine. Accordingly, it is possible to create a structural stabilization moiety using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al., J. Med. Chem., 2012, 55, 1137; PCT Publication No. WO 2010/060112). In addition, other methods of performing different types of stapling can be employed with the peptidomimetic compounds described herein (see, e.g., Lactam stapling: Shepherd et al., J. Am. Chem. Soc., 2005, 127, 2974-2983; UV cycloaddition stapling: Madden et al., Bioorg. Med. Chem. Lett., 2011, 21, 1472-1475; Disulfide stapling: Jackson et al., Am. Chem. Soc., 1991, 113, 9391-9392; Oxime stapling: Haney et al., Chem. Commun., 2011, 47, 10915-10917; Thioether stapling: Brunel and Dawson, Chem. Commun., 2005, 552-2554; Photoswitchable stapling: Kumita et al., Proc. Natl. Acad. Sci. U S. A., 2000, 97, 3803-3808; Double-click stapling: Lau et al., Chem. Sci., 2014, 5, 1804-1809; Bis-lactam stapling: Phelan et al., J. Am. Chem. Soc., 1997, 119, 455-460; and Bis-arylation stapling: Spokoyny et al., J. Am. Chem. Soc., 2013, 135, 5946-5949).

In some embodiments, the structural stabilization moiety comprises a ring closing metathesis moiety, a copper catalyzed azide alkyne cycloaddition moiety, a lactamization reaction moiety, a cysteine-xylene stapling moiety, a cysteine-perfluorobenzene stapling moiety, a hiol-yne/-ene click chemistry group, a selenocysteine stapling moiety, a tryptophan condensation moiety, a C—H activation moiety, or a 1,3-dipolar cycloaddition stapling moiety. In some embodiments, the structural stabilization moiety comprises a ring closing metathesis moiety. In some embodiments, the structural stabilization moiety comprises a copper catalyzed azide alkyne cycloaddition moiety. In some embodiments, the structural stabilization moiety comprises a lactamization reaction moiety. In some embodiments, the structural stabilization moiety comprises a cysteine-xylene stapling moiety. In some embodiments, the structural stabilization moiety comprises a cysteine-perfluorobenzene stapling moiety. In some embodiments, the structural stabilization moiety comprises a hiol-yne/-ene click chemistry group. In some embodiments, the structural stabilization moiety comprises a selenocysteine stapling moiety. In some embodiments, the structural stabilization moiety comprises a tryptophan condensation moiety. In some embodiments, the structural stabilization moiety comprises a C—H activation moiety. In some embodiments, the structural stabilization moiety comprises a 1,3-dipolar cycloaddition stapling moiety.

In some embodiments, the peptidomimetic compound is fused to a heterologous molecule. In some embodiments, the heterologous molecule comprises an immunoglobulin Fc domain, a peptide purification tag, a fluorescent protein, or a transduction domain. In some embodiments, the heterologous molecule comprises an immunoglobulin Fc domain. In some embodiments, the heterologous molecule comprises a peptide purification tag. In some embodiments, the heterologous molecule comprises a fluorescent protein. In some embodiments, the heterologous molecule comprises a transduction domain. Numerous purification tags are described in U.S. Pat. Nos. 8,426,566 and 8,357,511. Examples of protein tags include, but are not limited to, ALFA-tag, AviTag, C-tag, Calmodulin-tag, polyglutamate tag, polyarginine tag, E-tag, FLAG-tag, HA-tag, His-tag, Myc-Tag, NE-tag, Rho1D4-tag, S-tag, SBP-tag, Softag, Spot-tag, Strep-tag, T7-tag, TC-tag, Ty tag, V5-tag, VSV-tag, and Xpress-tag.

In some embodiments, heterologous molecule comprises a fluorescent protein. Examples of fluorescent proteins include, but are not limited to, green fluorescent protein (GFP; see, for example, GenBank Accession Number M62654) from the Pacific Northwest jellyfish, Aequorea Victoria and natural and engineered variants thereof (see, for example, U.S. Pat. Nos. 5,804,387, 6,090,919, 6,096,865, 6,054,321, 5,625,048, 5,874,304, 5,777,079, 5,968,750, 6,020,192, and 6,146,826, and PCT Publication NO. WO 99/64592). Other examples include Split-GFP, Split-YFP (described in U.S. Patent Application Publication No. US2012/0282643), Split-CFP (Id.) and Split-GFP variants, folding variants of GFP (e.g., more soluble versions, superfolder versions), spectral variants of GFP which have a different fluorescence spectrum (e.g., YFP, CFP), and GFP-like fluorescent proteins (e.g., DsRed; and DsRed variants, including DsRed1, DsRed2 (see, e.g., Matz et al., Nat. Biotechnol., 1999, 17, 969-973). Fluorescent proteins with distinct excitation and emission properties include, for example, functional GFPs, CFPs and YFPs that comprise distinct excitation and emission properties (see, e.g., Tsien, Annu. Rev. Biochem., 1998, 67, 509-544).

In some embodiments, the heterologous molecule comprises a transduction domain. Examples of transduction domains include, but are not limited to, peptides derived from the HIV Tat-derived peptide (Ruben et al., J. Virol., 1989, 63, 1-8), and peptides derived from Drosophila antennapedia transcription factor (ANTP) (Mann and Frankel, EMBO J., 1991, 10, 1733-1739). Numerous additional examples of transduction domains are set forth in U.S. Pat. No. 8,980,843.

In some embodiments, the peptidomimetic compound is linked to a label. In some embodiments, the label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. In some embodiments, the label is a fluorescent label or a radiolabel. In some embodiments, the label is a fluorescent label. In some embodiments, the label is a radiolabel. Such labels include, for example, radiolabels (e.g., ¹³N, ¹⁸F, ³²P, ⁶⁴Cu, ⁶⁶Ga, ⁶⁷Ga, ⁶⁸Ga, ⁶⁷Cu, ⁷⁷Br, ^{80m}Br, ⁸²Rb, ⁸⁶Y, ⁹⁰Y, ⁹⁵Ru, ⁹⁷Ru, ^{99m}Tc, ¹⁰³Ru, ¹⁰⁵Ru, ¹¹¹In, ^{113m}In, ¹¹³Sn, ^{121m}Te, ^{122m}Te, ^{125m}Te, ¹²³I, ¹²⁴I, ¹²⁵I, ¹²⁶I, ¹³¹I, ¹³³I, ¹⁶⁵Tm, ¹⁶⁷Tm, ¹⁶⁸Tm, ¹⁷⁷Lu, ¹⁸⁶Re, ¹⁸⁸Re, ^{195m}Hg, ²¹¹At, ²¹²Bi, ²¹³Bi, and ²²⁵Ac), pigments, dyes, chromogens, spin labels, and fluorescent labels (e.g., fluorescein, fluorescein isothiocyanate, rhodamine, 5-dimethylamine-1-napthalenesulfonyl chloride, phycoerythrin and the like). The label can also be, for example, a chemiluminescent substance, a metal-containing substance, or an enzyme (e.g. alkaline phosphatase, horseradish peroxidase, glucose oxidase, and the like), where there occurs an enzyme-dependent secondary generation of signal. The label can also be a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

In some embodiments, the label comprises polyethylene glycol, polysialic acid, or glycolic acid. In some embodiments, the label comprises polyethylene glycol. In some embodiments, the label comprises polysialic acid. In some embodiments, the label comprises glycolic acid.

In some embodiments, the peptidomimetic compounds described herein bind to TLN1 with a K_{A} (kₒₙ/k_{off}) of at least about 10¹⁰ M⁻¹, at least about 4×10¹¹ M⁻¹, at least about 10¹¹ M⁻¹, at least about 4×10¹² M⁻¹, at least about 10¹² M⁻¹, at least about 4×10¹³ M⁻¹, at least about 10¹³ M⁻¹, at least about 4×10¹⁴ M⁻¹, at least about 10¹⁴ M⁻¹, at least about 4×10¹⁵ M⁻¹, or at least about 10¹⁵ M⁻¹, or with a K_{A} of any range from and to any pair of the foregoing values (such as, from about 4×10¹¹ M⁻¹ to about 4×10¹³ M⁻¹ or from about 4×10¹² M⁻¹ to about 4×10¹⁵ M⁻¹).

In some embodiments, the peptidomimetic compounds described herein bind to TLN1 with a K_{D} (k_{off}/kₒₙ) of about 10⁻¹⁰ or less, about 4×10⁻¹¹ M or less, about 10⁻¹¹ M or less, about 4×10⁻¹² M or less, about 10⁻¹² M or less, about 4×10¹³ M or less, about 10⁻¹³ M or less, about 4×10¹⁴ M or less, about 10⁻¹⁴ M or less, about 4×10⁻¹⁵ M or less, or about 10⁻¹⁵ M or less, or with a K_{D} of any range from and to any pair of the foregoing values (such as, from about 4×10⁻¹¹ M to about 4×10⁻¹³ M or from about 4×10⁻¹² M to about 4×10⁻¹⁵ M). In some embodiments, the K_{D} (k_{off}/kₒₙ) value is determined by assays, such as ELISA, isothermal titration calorimetry (ITC), fluorescent polarization assay or any other biosensors such as BIAcore.

In some embodiments, the peptidomimetic compounds described herein bind to TLN1 and inhibit the binding of TLN1 and integrin at an IC₅₀ of less than about 0.02 nM, less than about 0.01 nM, less than about 0.005 nM, less than about 0.002 nM, less than about 0.001 nM, less than about 5×10⁻⁴ nM, less than about 2×10⁻⁴ nM, less than about 1×10⁻⁴ nM, less than about 5×10⁻⁵ nM, less than about 2×10⁻⁵ nM, less than about 1×10⁻⁴ nM, less than about 5×10⁻⁶ nM, less than about 2×10⁻⁶ nM, less than about 1×10⁻⁶ nM, less than about 5×10⁻⁷ nM, less than about 2×10⁻⁷ nM, or less than about 1×10⁻⁷ nM, or with an IC₅₀ of any range from and to any pair of the foregoing values (such as, from about 0.02 nM to about 2×10⁻⁵ nM, or from about 5×10⁻⁵ nM to about 1×10⁻⁷ nM). The IC₅₀ can be measured according to methods such as ELISA.

The present disclosure also comprises compositions comprising any of the peptidomimetic compounds described herein and a carrier or excipient. In some embodiments, the peptidomimetic compounds retain their structure under physiological conditions, such as in the body of a subject (e.g., in the gastrointestinal tract or bloodstream). In some embodiments, the composition is a pharmaceutical composition. Such pharmaceutical compositions may optionally comprise one or more additional biologically active substances.

Although the pharmaceutical compositions provided herein are principally directed to pharmaceutical compositions which are suitable for administration to humans, such compositions are generally suitable for administration to animals of all sorts. Subjects to which administration of the pharmaceutical compositions is contemplated include, but are not limited to, humans and/or other primates, mammals, including commercially relevant mammals such as cattle, pigs, horses, sheep, cats, and/or dogs, and/or birds, including commercially relevant birds, such as chickens, ducks, geese, and/or turkeys.

The formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage.

The relative amounts of the active ingredient, the pharmaceutically acceptable excipient, and/or any additional ingredients in a pharmaceutical composition will vary, depending upon the identity, size, and/or condition of the subject treated and further depending upon the route by which the composition is to be administered. By way of example, the composition may comprise between 0.1% and 100% (w/w) of the active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and any combination thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and any combination thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or any combination thereof.

Exemplary binding agents include, but are not limited to, starch (e.g., cornstarch and starch paste), gelatin, sugars (e.g., sucrose, glucose, dextrose, dextrin, molasses, lactose, lactitol, mannitol), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinylpyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and any combination thereof.

Preservatives can be added to retard microbial growth, and can be added in amounts ranging from 0.2%-1% (w/v). Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In some embodiments, the preservative is an anti-oxidant. In some embodiments, the preservative is a chelating agent.

Buffering agents help to maintain the pH in the range that approximates physiological conditions. Buffering agents can be present at concentration ranging from about 2 mM to about 50 mM. Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and any combination thereof.

Non-ionic surfactants or detergents (also known as “wetting agents”) can also be added to pharmaceutical compositions to help solubilize the peptidomimetic compounds described herein as well as to protect the peptidomimetic compounds against agitation-induced aggregation, which also permits the formulation to be exposed to shear surface stressed without causing denaturation of the proteins. Exemplary non-ionic surfactants include, but are not limited to, polysorbates (20, 80, etc.), polyoxamers (184, 188, etc.). Pluronic polyols, polyoxyethylene sorbitan monoethers (TWEEN™-20, TWEEN™-80, and the like). Nonionic surfactants can be present in a range of about 0.05 mg/mL to about 1.0 mg/mL, for example, about 0.07 mg/mL to about 0.2 mg/mL.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and any combination thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, Eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and any combination thereof.

Exemplary carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In some embodiments for parenteral administration, the peptidomimetic compounds are mixed with solubilizing agents such as CREMOPHOR, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

To prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternately, delayed absorption of a parenterally administered drug form is accomplished by dissolving or suspending the drug in an oil vehicle.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It may be especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms are dictated by and directly dependent on the unique characteristics of the active compound and the particular therapeutic effect to be achieved, and the limitations inherent in the art of compounding such an active compound for the treatment of individuals.

Compositions for rectal or vaginal administration are typically suppositories which can be prepared by mixing the peptidomimetic compounds with suitable non-irritating excipients, such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polethylene glycols and the like.

The active ingredients can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a peptidomimetic compound may include ointments, pastes, creams, lotions, gels, powders, solutions, sprays, inhalants and/or patches. Generally, the active component is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of an active ingredient to the body. Such dosage forms may be prepared, for example, by dissolving and/or dispensing the active ingredient in the proper medium. Alternately or additionally, the rate may be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499, 5,190,521, 5,328,483, 5,527,288, 4,270,537, 5,015,235, 5,141,496, and 5,417,662. Intradermal compositions may be administered by devices which limit the effective penetration length of a needle into the skin, such as those described in PCT Publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, and PCT Publications WO 97/37705 and WO 97/13537. Ballistic powder/particle delivery devices which use compressed gas to accelerate vaccine in powder form through the outer layers of the skin to the dermis are suitable. Alternately or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

The formulations described herein as being useful for pulmonary delivery are useful for intranasal delivery of a pharmaceutical composition. Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1 to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are contemplated as being within the scope of the present disclosure.

A pharmaceutical composition may be prepared, packaged, and/or sold in a formulation suitable storage as lyophilized formulations.

The present disclosure also provides methods of producing the peptidomimetic compounds described herein. In some embodiments, the methods comprise culturing a host cell comprising a nucleic acid molecule encoding a peptide comprising the amino acid sequence NEDIDQMFSTLLGEMDLLTQS (SEQ ID NO:1) having from 0 to 5 conservative amino acid substitutions, modifications, deletions, or any combination thereof, whereby the host cell expresses the peptide. In some embodiments, the methods further comprise recovering the expressed peptide from the host cell culture. In some embodiments, the methods further comprise linking two non-adjacent amino acids within SEQ ID NO:1 with at least one structural stabilization moiety.

In some embodiments, the methods further comprise conjugating the peptidomimetic compound to a heterologous molecule or a label. In some embodiments, the methods further comprise conjugating the peptidomimetic compound to a heterologous molecule. In some embodiments, the methods further comprise conjugating the peptidomimetic compound to a label.

In some embodiments, the nucleic acid molecule is under the control of a heterologous promoter. In some embodiments, the nucleic acid molecule is under the control of an inducible promoter.

In some embodiments, the peptidomimetic compounds described herein can inhibit the interaction between Talin-1 (TLN1) and integrin. Without desiring to be bound by any particular theory, it is believed that the interaction between RIAM TBS and the TLN1 ROD domain stabilizes the autoinhibitory conformation of TLN1, where the TLN1 head domain is bound to TLN1 ROD domain, thus preventing the TLN1 head domain from interacting with integrin and activating the “inside-out” signaling mechanism. Accordingly, it is believed that a moiety interacting with TLN1 ROD domain in a manner that functionally reproduces TLN1-RIAM interaction can likewise stabilize the autoinhibitory TLN1 conformation thereby effectively acting as the inhibitor of TLN1-Integrin interaction.

The present disclosure also provides pharmaceutical packs or kits comprising one or more containers filled with one or more of the ingredients of the pharmaceutical compositions described herein. Optionally associated with such container(s) is a notice in the form prescribed by a governmental agency regulating the manufacture, use or sale of pharmaceuticals or biological products, which notice reflects approval by the agency of manufacture, use or sale for human administration.

The synthesis of the peptidomimetic compounds described herein can involve 1) synthesizing a peptide from a selected number of natural or non-natural amino acids, wherein said peptide comprises at least two reactive moieties capable of undergoing a C—C bond forming reaction; and 2) contacting the peptide with a reagent to generate at least one crosslinker and to effect stabilization of a specific secondary structure motif (e.g., an α-helix).

The number, stereochemistry, and type of amino acid structures (natural or non-natural) selected can depend upon the size and shape of the secondary structure to be prepared (e.g., length of an α-helix), and the ability of the particular amino acids to generate a secondary structural motif that are desirable to mimic. The secondary structure to be prepared depends on the desired biological activity—the ability to target an effector biomolecule or a target biomolecule with an affinity sufficient to be specific and to follow the two biomolecules together.

Once the amino acids are selected, synthesis of the peptidomimetic compounds can be achieved using standard deprotection and coupling reactions. Formation of peptide bonds and polypeptide synthesis encompass both solid phase and solution phase methods; see generally, Bodanszky and Bodanszky, The Practice of Peptide Synthesis, Springer-Verlag, Berlin, 1984; Atherton and Sheppard, Solid Phase Peptide Synthesis: A Practical Approach, IRL Press at Oxford University Press Oxford, England, 1989, and Stewart and Young, Solid phase Peptide Synthesis, 2nd edition, Pierce Chemical Company, Rockford, 1984. In both solution phase and solid phase techniques, the choice of the protecting groups can be considered, as well as the specific coupling techniques to be utilized. For a detailed discussion of peptide synthesis techniques for solution phase and solid phase reactions, see, Bioorganic chemistry: Peptides and Proteins, Hecht, Oxford University Press, New York: 1998.

In some embodiments, the methods comprise a solution phase synthesis of peptidomimetic compounds. An exemplary solution phase synthesis comprises the steps: 1) providing an amino acid protected at the N-terminus with a suitable amino protecting group; 2) providing an amino acid protected at the C-terminus with a suitable carboxylic acid protecting group; 3) coupling the N-protected amino acid to the C-protected amino acid; 4) deprotecting the product of the coupling reaction; and 5) repeating steps 3) to 4) until a desired polypeptide is obtained, wherein at least two of the amino acids coupled at any of the above steps each comprise at least one terminally unsaturated amino acid side chain, and at least one α,α-disubstituted amino acid comprises two terminally unsaturated amino acid side chains. During the course of the above synthesis, various parameters can be varied including, but not limited to, placement of amino acids with terminally unsaturated side chains, stereochemistry of amino acids, terminally unsaturated side chain length and functionality, and amino acid residues utilized.

In some embodiments, the methods comprise a solid phase synthesis of peptidomimetic compounds. An exemplary solid phase synthesis comprises the steps: 1) providing a resin-bound amino acid; 2) deprotecting the resin bound amino acid; 3) coupling an amino acid to the deprotected resin-bound amino acid; 4) repeating step 3) until a desired peptide is obtained, wherein at least two of the amino acids coupled at any of the above steps each comprise at least one terminally unsaturated amino acid side chain, and at least one α,α-disubstituted amino acid comprises two terminally unsaturated amino acid side chains. During the course of the above synthesis, various parameters can be varied including, but not limited to, placement of amino acids with terminally unsaturated side chains, stereochemistry of amino acids, terminally unsaturated side chain length and functionality, and amino acid residues utilized.

In some embodiments, the peptide is biologically produced by culturing in a suitable medium, a host cell (e.g., a prokaryotic or eukaryotic host cell) containing a recombinant expression vector such that the protein is produced.

Recombinant expression vectors can be designed for expression of peptidomimetic compounds in prokaryotic or eukaryotic cells, e.g., for use in the methods described herein. For example, peptidomimetic compounds can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, CA (1990). Alternately, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility and/or stability of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), μMAL (New England Biolabs, Beverly, MA) and pRIT5 (Pharmacia, Piscataway, NJ) which fuse glutathione S-transferase (GST), maltose E binding protein, or protein A, respectively, to the target recombinant protein.

Examples of suitable inducible non-fusion E. coli expression vectors include pTrc (Amann et al, Gene, 1988, 69, 301-315) and pET lid (Studier et al, Gene Expression Technology: Methods in Enzymology 185, Academic Press, San Diego, California (1990) 60-89). Target gene expression from the pTrc vector relies on host RNA polymerase transcription from a hybrid trp-lac fusion promoter. Target gene expression from the pET lid vector relies on transcription from a T7 gnlO-lac fusion promoter mediated by a coexpressed viral RNA polymerase (T7 gnl). This viral polymerase is supplied by host strains BL21(DE3) or HMS174(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, California (1990) 119-128). Another strategy is to alter the nucleic acid sequence of the nucleic acid to be inserted into an expression vector so that the individual codons for each amino acid are those preferentially utilized in E. coli (Wada et al., Nuc. Acids Res., 1992, 20, 2111-2118). Such alteration of nucleic acid sequences can be carried out by standard DNA synthesis techniques.

In some embodiments, the expression vector is a yeast expression vector. Examples of vectors for expression in yeast S. cerevisiae include pYepSecl (Baldari et al, EMBO J., 1987, 6, 229-234), pMFa (Kurjan and Herskowitz, Cell, 1982, 30, 933-943), pJRY88 (Schultz et al, Gene, 1987, 54, 113-123), pYES2 (Invitrogen Corporation, San Diego, CA), and picZ (InVitrogen Corp, San Diego, CA).

Alternately, peptidomimetic compounds can be expressed in insect cells using baculovirus expression vectors. Baculovirus vectors available for expression of proteins in cultured insect cells (e.g., Sf 9 cells) include the pAc series (Smith et al., Mol. Cell Biol., 1983, 3, 2156-2165) and the pVL series (Lucklow and Summers, Virology, 1989, 170, 31-39).

In some embodiments, a nucleic acid molecule is expressed in mammalian cells using a mammalian expression vector. Examples of mammalian expression vectors include pCDM8 (Seed, Nature, 1987, 329, 840) and pMT2PC (Kaufman et al., EMBO J., 1987, 6, 187-195). When used in mammalian cells, the expression vector's control functions are often provided by viral regulatory elements. For example, commonly used promoters are derived from polyoma, Adenovirus 2, cytomegalo virus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, NY, 1989. In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Examples of suitable tissue-specific promoters include, but are not limited to, the albumin promoter (liver-specific; Pinkert et al., Genes Dev., 1987, 1, 268-277), lymphoid-specific promoters (Calame and Eaton, Adv. Immunol., 1988, 43, 235-275), in particular promoters of T cell receptors (Winoto and Baltimore, EMBO J., 1989, 8, 729-733) and immunoglobulins (Banerji et al., Cell, 1983, 33, 729-740; Queen and Baltimore, Cell, 1983, 33, 741-748), neuron-specific promoters (e.g., the neurofilament promoter; Byrne and Ruddle, Proc. Natl. Acad. Sci. USA, 1989, 86, 5473-5477), endothelial cell-specific promoters (e.g., KDR/flk promoter; U.S. Pat. No. 5,888,765), pancreas-specific promoters (Edlund et al., Science, 1985, 230, 912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Grass, Science, 1990, 249, 374-379) and the α-fetoprotein promoter (Campes and Tilghman, Genes Dev., 1989, 3, 537-546).

A host cell can be any prokaryotic or eukaryotic cell. For example, peptidomimetic compounds can be expressed in bacterial cells such as E. coli, insect cells, yeast or mammalian cells (such as hematopoietic cells, leukocytes, K562 cells, 293T cells, human umbilical vein endothelial cells (HUVEC), human microvascular endothelial cells (HMVEC), Chinese hamster ovary cells (CHO) or COS cells).

Vector DNA can be introduced into prokaryotic or eukaryotic cells via conventional transformation or transfection techniques including, but not limited to, calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, NY, 1989), and other laboratory manuals.

For stable transfection of mammalian cells, to identify and select integrants, a gene that encodes a selectable marker (e.g., resistance to antibiotics) is generally introduced into the host cells along with the gene of interest. Suitable selectable markers include those which confer resistance to drugs, such as G418, hygromycin and methotrexate. Nucleic acids encoding a selectable marker can be introduced into a host cell on the same vector as that encoding peptidomimetic compounds or can be introduced on a separate vector. Cells stably transfected with the introduced nucleic acid can be identified by drug selection (e.g., cells that have incorporated the selectable marker gene will survive, while the other cells die).

After a desired polypeptide is synthesized using an appropriate technique, the polypeptide is contacted with a specific catalyst to promote stapling or stitching of the polypeptide. For example, the resin-bound polypeptide may be contacted with a catalyst to promote stapling or stitching, or may first be cleaved from the resin, and then contacted with a catalyst to promote stitching.

Different amino acids have different propensities for forming different secondary structures. For example, methionine (M), alanine (A), leucine (L), glutamate (E), and lysine (K) all have especially high alpha-helix forming propensities. In contrast, proline (P) and glycine (G) are alpha-helix disruptors.

In some embodiments, the one or more reaction steps further comprise the use of a coupling reagent. Exemplary coupling reagents include, but are not limited to, benzotriazol-1-yloxy-tris(dimethylamino)-phosphonium hexafluorophosphate (BOP), benzotriazole-1-yl-oxy-tris-pyrrolidino-phosphonium hexafluorophosphate (PyBOP), bromo-tris-pyrrolidino phosphonium hexafluorophosphate (PyBroP), 1-ethyl-3-(3-dimethyllaminopropyl) carbodiimide (EDC), N,N′-carbonyldiimidazole (CDI), 3-(diethoxyphosphoryloxy)-1,2,3-benzotriazin-4(3H)-one (DEPBT), 1-hydroxy-7-azabenzotriazole (HOAt), 1-hydroxy-7-benzotriazole (HOBt), 2-(7-aza-1H-benzotriazole-1-yl)-1,1,3,3-tetramethyluronium hexafluorophosphate (HATU), 2-(6-chloro-1H-benzotriazole-1-yl)-1,1,3,3-tetramethylaminium hexafluorophosphate (HCTU), 2-(1H-benzotriazole-1-yl)-1,1,3,3-tetramethyluronium hexafluorophosphate (HBTU), 0-(7-azabenzotriazole-1-yl)—N,N,N′,N′-tetramethyluronium tetrafluoroborate (TATU), 2-(1H-benzotriazole-1-yl)-1,1,3,3-tetramethyluronium tetrafluoroborate (TBTU), N,N,N′,N′-tetramethyl-O-(3,4-dihydro-4-oxo-1,2,3-benzotriazin-3-yl)uranium tetrafluoroborate (TDBTU), and O-(N-succinimidyl)-1,1,3,3-tetramethyl uranium tetrafluoroborate (TSTU)).

In some embodiments, the above reaction further comprises a suitable base. Suitable bases include, but are not limited to, potassium carbonate, potassium hydroxide, sodium hydroxide, tetrabutylammonium hydroxide, benzyltrimethylammonium hydroxide, triethylbenzylammonium hydroxide, 1,1,3,3-tetramethylguanidine, 1,8-diazabicyclo[5.4.0]undec-7-ene (DBU), N-methylmorpholine, diisopropylethylamine (DIPEA), tetramethylethylenediamine (TMEDA), pyridine (Py), 1,4-diazabicyclo [2.2.2]octane (DABCO), N,N-dimethylamino pyridine (DMAP), or triethylamine (NEt3).

In some embodiments, one or more reaction steps are carried out in a suitable medium. A suitable medium is a solvent or a solvent mixture that, in combination with the combined reacting partners and reagents, facilitates the progress of the reaction there between. A suitable solvent may solubilize one or more of the reaction components, or, alternatively, the suitable solvent may facilitate the suspension of one or more of the reaction components; see generally, March's Advanced Organic Chemistry: Reactions, Mechanisms, and Structure, M. B. Smith and J. March, 5^{th} Edition, John Wiley & Sons, 2001, and Comprehensive Organic Transformations, R. C. Larock, 2^{nd} Edition, John Wiley & Sons, 1999. Suitable solvents include ethers, halogenated hydrocarbons, aromatic solvents, polar aprotic solvents, or mixtures thereof. In other embodiments, the solvent is diethyl ether, dioxane, tetrahydrofuran (THF), dichloromethane (DCM), dichloroethane (DCE), acetonitrile (ACN), chloroform, toluene, benzene, dimethylformamide (DMF), dimethylacetamide (DMA), dimethylsulfoxide (DMSO), N-methyl pyrrolidinone (NMP), or any mixture thereof.

In some embodiments, one or more reaction steps are conducted at suitable temperature, such as between about 0° C. and about 100° C.

In some embodiments, one or more reaction steps involve a catalyst. Selection of a particular catalyst can vary with the reaction conditions utilized and the functional groups present in the particular peptide. In some embodiments, the catalyst is a ring closing metathesis (RCM) catalyst. In some embodiments, the RCM catalyst is a tungsten (W), molybdenum (Mo), or ruthenium (Ru) catalyst. In some embodiments, the RCM catalyst is a ruthenuim catalyst. Suitable RCM catalysts are described in Grubbs et al., Acc. Chem. Res., 1995, 28, 446-452; U.S. Pat. No. 5,811,515; Schrock et al., Organometallics, 1982, 1, 1645; Gallivan et al., Tetrahedron Letters, 2005, 46, 2577-2580; Furstner et al., J. Am. Chem. Soc., 1999, 121, 9453; and Chem. Eur. J., 2001, 7, 5299. In some embodiments, the RCM catalyst is a Schrock catalyst, a Grubbs catalyst, a Grubbs-Hoveyda catalyst, a Blechart Catalyst; a Neolyst™ Ml; or a Furstner catalyst.

In addition to RCM catalysts, other reagents capable of promoting carbon-carbon bond formation can also be utilized. For example, other reactions that can be utilized, include, but are not limited to palladium coupling reactions, transition metal catalyzed cross coupling reactions, pinacol couplings (terminal aldehydes), hydrozirconation (terminal alkynes), nucleophilic addition reactions, and NHK (Nozaki-Hiyama-Kishi) (Furstner et al., J. Am. Chem. Soc., 1996, 118, 12349)) coupling reactions. Appropriate reactive moieties are first incorporated into desired amino acids or unnatural amino acids, and then the peptide is subjected to reaction conditions to effect stapling or stitching and subsequent stabilization of a desired secondary structure.

Additional methods of producing stapled or stitched polypeptides are described, for example, in PCT Publications WO 2004/007530 and WO 2011/008260.

The present disclosure provides methods of treating a subject having an integrin-related disorder. The methods comprise administering a peptidomimetic compound comprising the amino acid sequence NEDIDQMFSTLLGEMDLLTQS (SEQ ID NO:1) having from 0 to 5 conservative amino acid substitutions, modifications, deletions, or any combination thereof, or a pharmaceutically acceptable salt thereof. The compound comprises at least one structural stabilization moiety. The peptidomimetic compound can be any of the peptidomimetic compounds described herein. In some embodiments, the peptidomimetic compound is present within a composition comprising a pharmaceutically acceptable carrier.

In some embodiments, the at least one structural stabilization moiety links two nonadjacent amino acid residues within SEQ ID NO: 1. In some embodiments, the at least one structural stabilization moiety links any two non-adjacent amino acids within positions 10 to 21 of SEQ ID NO:1. In some embodiments, the at least one structural stabilization moiety links any two non-adjacent amino acids within positions 1 to 9 of SEQ ID NO:1. In some embodiments, the at least one structural stabilization moiety links any amino acid except 14, F8, L11, and L18 to another non-adjacent amino acid within SEQ ID NO:1. In some embodiments, the two nonadjacent amino acid residues within SEQ ID NO:1 are 2 to 7 amino acids apart. In some embodiments, the two nonadjacent amino acid residues within SEQ ID NO:1 are 2 amino acids apart. In some embodiments, the two nonadjacent amino acid residues within SEQ ID NO:1 are 3 amino acids apart. In some embodiments, the two nonadjacent amino acid residues within SEQ ID NO:1 are 4 amino acids apart. In some embodiments, the two nonadjacent amino acid residues within SEQ ID NO:1 are 5 amino acids apart. In some embodiments, the two nonadjacent amino acid residues within SEQ ID NO:1 are 6 amino acids apart. In some embodiments, the two nonadjacent amino acid residues within SEQ ID NO:1 are 7 amino acids apart. In some embodiments, the at least one structural stabilization moiety links the amino acid at position 15 to the amino acid at position 19 of SEQ ID NO:1.

In some embodiments, the structural stabilization moiety is a hydrocarbon having from about 5 to about 20 carbons. In some embodiments, the hydrocarbon comprises a substituted or unsubstituted alkane comprising from about 5 to about 20 carbons, a substituted or unsubstituted alkene comprising from about 5 to about 20 carbons, or a substituted or unsubstituted alkyne comprising from about 5 to about 20 carbons. In some embodiments, the structural stabilization moiety is a hydrocarbon having from about 8 to about 12 carbons. In some embodiments, the hydrocarbon comprises a substituted or unsubstituted alkane comprising from about 8 to about 12 carbons, a substituted or unsubstituted alkene comprising from about 8 to about 12 carbons, or a substituted or unsubstituted alkyne comprising from about 8 to about 12 carbons. In some embodiments, the hydrocarbon comprises a substituted or unsubstituted alkane comprising about 8 carbons, a substituted or unsubstituted alkene comprising about 8 carbons, or a substituted or unsubstituted alkyne comprising about 8 carbons.

In some embodiments, the structural stabilization moiety comprises a ring closing metathesis moiety, a copper catalyzed azide alkyne cycloaddition moiety, a lactamization reaction moiety, a cysteine-xylene stapling moiety, a cysteine-perfluorobenzene stapling moiety, a hiol-yne/-ene click chemistry group, a selenocysteine stapling moiety, a tryptophan condensation moiety, a C—H activation moiety, or a 1,3-dipolar cycloaddition stapling moiety.

In some embodiments, the peptidomimetic compound is fused to a heterologous molecule. In some embodiments, the heterologous molecule comprises an immunoglobulin Fc domain, a peptide purification tag, a fluorescent protein, or a transduction domain. In some embodiments, the peptidomimetic compound is linked to a label. In some embodiments, the label is a fluorescent label or a radiolabel. In some embodiments, the label comprises polyethylene glycol, polysialic acid, or glycolic acid.

In some embodiments, the integrin-related disorder is a thrombotic disorder, a cardiovascular disease, an autoimmune disease, or a cancer. In some embodiments, the integrin-related disorder is a thrombotic disorder. In some embodiments, the integrin-related disorder is a cardiovascular disease. In some embodiments, the integrin-related disorder is an autoimmune disease. In some embodiments, the integrin-related disorder is a cancer. In some embodiments, the integrin-related disorder is also a talin-related disorder.

In some embodiments, the thrombotic disorder is angina or restenosis. In some embodiments, the thrombotic disorder is angina. In some embodiments, the thrombotic disorder is restenosis.

In some embodiments, the cardiovascular disease is stroke or acute coronary artery disease. In some embodiments, the cardiovascular disease is stroke. In some embodiments, the cardiovascular disease is acute coronary artery disease.

In some embodiments, the autoimmune disease is asthma, psoriasis, multiple sclerosis, ulcerative colitis, rheumatoid arthritis, or Crohn's disease. In some embodiments, the autoimmune disease is asthma. In some embodiments, the autoimmune disease is psoriasis. In some embodiments, the autoimmune disease is multiple sclerosis. In some embodiments, the autoimmune disease is ulcerative colitis. In some embodiments, the autoimmune disease is rheumatoid arthritis. In some embodiments, the autoimmune disease is Crohn's disease.

In some embodiments, the cancer is renal cell carcinoma, metastatic melanoma, or pancreatic cancer. In some embodiments, the cancer is renal cell carcinoma. In some embodiments, the cancer is metastatic melanoma. In some embodiments, the cancer is pancreatic cancer.

Administration of the peptidomimetic compounds may be for either a prophylactic or therapeutic purpose. In some embodiments, a therapeutically effective dose refers to that amount of a peptidomimetic compound sufficient to result in a detectable change in the physiology of a recipient subject. In some embodiments, a therapeutically effective dose refers to an amount of a peptidomimetic compound sufficient to result in modulation of an inflammatory and/or immune response, thrombosis, cell adhesion, or cellular proliferation, growth, or migration. In some embodiments, a therapeutically effective dose refers to an amount of a peptidomimetic compound sufficient to result in the amelioration of symptoms of a thrombotic disorder, a cardiovascular disease, an autoimmune disease, or a cancer. In some embodiments, a therapeutically effective dose refers to an amount of a peptidomimetic compound sufficient to prevent a thrombotic disorder, a cardiovascular disease, an autoimmune disease, or a cancer. In some embodiments, a therapeutically effective dose refers to that amount of a peptidomimetic compound sufficient to modulate talin 1 activity (e.g., a signaling activity or a ligand binding activity) as described herein. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are more suitable.

The dosage of peptidomimetic compound lies within a range of circulating concentrations that include the ED 50 with little or no toxicity. The dosage may vary within this range depending upon the dosage form employed and the route of administration utilized. For any peptidomimetic compound used in the methods described herein, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. A therapeutically effective amount of a peptidomimetic compound (i.e., an effective dosage) ranges from about 0.001 to about 30 mg/kg body weight, from about 0.01 to about 25 mg/kg body weight, from about 0.1 to about 20 mg/kg body weight, and from about 1 to about 10 mg/kg, from about 2 to about 9 mg/kg, from about 3 to about 8 mg/kg, from about 4 to about 7 mg/kg, or from about 5 to about 6 mg/kg body weight.

Factors may influence the dosage required to effectively treat a subject, including but not limited to, the severity of the disease or disorder, previous treatments, the general health and/or age of the subject, and other diseases present. Moreover, treatment of a subject with a therapeutically effective amount of a peptidomimetic compound can include a single treatment or can include a series of treatments. In some embodiments, a subject is treated with a peptidomimetic compound as described herein, in the range of from about 0.1 to about 20 mg/kg body weight, one time per week for about 1 to about 10 weeks, from about 2 to about 8 weeks, from about 3 to about 7 weeks, or from about 4, 5, or 6 weeks. The effective dosage of a peptidomimetic compound as described herein used for treatment may increase or decrease over the course of a particular treatment. Changes in dosage may result and become apparent from the results of diagnostic assays as described herein. In some embodiments, a subject is treated with an initial dosing of a therapeutically effective amount of a peptidomimetic compound as described herein which reacts with or binds TLN1 followed by a subsequent intermittent dosing of a therapeutically effective amount of the peptidomimetic compound that is less than 100%, calculated on a daily basis, of the initial dosing of the peptidomimetic compound wherein the peptidomimetic compound is administered not more than once per week during the subsequent dosing. In some embodiments, the subsequent dosing is two or more times per week. In some embodiments, the subsequent dosing is one or more time every two weeks. In some embodiments, the subsequent dosing is one or more times every three weeks. In some embodiments, the subsequent dosing is one or more times every four weeks.

In some embodiments, the subsequent dosing is less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%, calculated on a daily basis, of the initial dosing of the peptidomimetic compound. In some embodiments, the initial dosage is from about 0.001 to about 30 mg/kg body weight, from about 0.01 to about 25 mg/kg body weight, from about 0.1 to about 20 mg/kg body weight, or from about 1 to about 10 mg/kg, from about 2 to about 9 mg/kg, from about 3 to about 8 mg/kg, from about 4 to about 7 mg/kg, or from about 5 to about 6 mg/kg body weight. In some embodiments, the initial dosage is less than about 0.3 mg/kg body weight, e.g., from about 0.001 to about 0.30 mg/kg body weight, e.g., about 0.1 mg/kg body weight, about 0.125 mg/kg body weight, about 0.15 mg/kg body weight, about 0.175 mg/kg body weight, about 0.2 mg/kg body weight, about 0.225 mg/kg body weight, about 0.25 mg/kg body weight, or about 0.275 mg/kg body weight.

In some embodiments, a subject is treated with an initial dosing of a therapeutically effective amount of a peptidomimetic compound as described herein which reacts with or binds TLN1, followed by a subsequent intermittent dosing of a therapeutically effective amount of the peptidomimetic compound that is greater than 100%, calculated on a daily basis, of the initial dosing of the peptidomimetic compound wherein the peptidomimetic compound is administered to the mammal not more than once per week during the subsequent dosing. In some embodiments, the subsequent dosing is two or more times per week. In some embodiments, the subsequent dosing is one or more time every two weeks. In some embodiments, the subsequent dosing is one or more times every three weeks. In some embodiments, the subsequent dosing is one or more times every four weeks. In some embodiments, the initial dosage is from about 0.001 to about 30 mg/kg body weight, from about 0.01 to about 25 mg/kg body weight, from about 0.1 to about 20 mg/kg body weight, or from about 1 to about 10 mg/kg, from about 2 to about 9 mg/kg, from about 3 to about 8 mg/kg, from about 4 to about 7 mg/kg, or from about 5 to about 6 mg/kg body weight. In some embodiments, the initial dosage is less than about 0.3 mg/kg body weight, e.g., from about 0.001 to about 0.3 mg/kg body weight, e.g., about 0.1 mg/kg body weight, about 0.125 mg/kg body weight, about 0.15 mg/kg body weight, about 0.175 mg/kg body weight, about 0.2 mg/kg body weight, about 0.225 mg/kg body weight, about 0.25 mg/kg body weight, or about 0.275 mg/kg body weight.

In some embodiments, an initial dosage is followed by the same dosage, for example, not more than once per week during the subsequent dosing. In some embodiments, the subsequent dosing is two or more times per week. In some embodiments, the subsequent dosing is one or more time every two weeks. In some embodiments, the subsequent dosing is one or more times every three weeks. In some embodiments, the subsequent dosing is one or more times every four weeks.

The peptidomimetic compounds and pharmaceutical compositions described herein can be administered to a subject via any route, e.g., any route approved by the Food and Drug Administration (FDA). Suitable routes of administration include, but are not limited to, oral, intravenous, intramuscular, intra-arterial, intramedullary, intraspinal, intracranial, intraocular, intrathecal, intracerebroventricular, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. Specific contemplated routes are systemic injection (intraarterial or intravenous), regional administration via blood and/or lymph supply, and/or direct administration to an affected site. In general, the most appropriate route of administration will depend upon a variety of factors including the nature of the agent (e.g., its stability in the environment of the gastrointestinal tract), and the condition of the subject (e.g., whether the subject is able to tolerate oral administration). One or more peptidomimetic compounds may be administered simultaneously by the same or different routes, or at different times during treatment. The peptidomimetic compounds may also be prescribed to be taken in combination with other drugs used to treat a thrombotic disorder, a cardiovascular disease, an autoimmune disease, or a cancer. When used in such combinations, peptidomimetic compounds and conventional drugs may be administered simultaneously, by the same or different routes, or at different times during treatment. When administered adjunctive to or with other agents, such as other chemotherapeutic agents, the peptidomimetic compounds described herein can be administered on the same schedule as the other agent(s), or on a different schedule. When administered on the same schedule, peptidomimetic compounds described herein can be administered before, after, or concurrently with the other agent. In some embodiments, where the peptidomimetic compounds described herein are administered adjunctive to, or with, standards of care, peptidomimetic compounds can be initiated prior to commencement of the standard therapy, for example one day, several days, one week, several weeks, one month, or even several months before commencement of standard of care therapy. In some embodiments, where the peptidomimetic compounds described herein are administered adjunctive to, or with, standards of care, peptidomimetic compounds described herein can be initiated after commencement of the standard therapy, for example one day, several days, one week, several weeks, one month, or even several months after commencement of standard of care therapy. The dose of the standard of care therapy selected will depend on the particular compound being used and the route and frequency of administration.

The treatment may be carried out for as long a period as necessary. Typically, it is contemplated that treatment would be continued indefinitely while the disease state persists, although discontinuation might be indicated if the compounds no longer produce a beneficial effect. The treating physician will know how to increase, decrease, or interrupt treatment based on patient response.

The therapeutic effect can comprise one or more of a decrease/reduction in symptoms of an integrin-related disorder, a decrease/reduction in the severity of the integrin-related disorder, a decrease/reduction in symptoms and integrin-related disorder-related effects, delaying the onset of symptoms and integrin-related disorder-related effects, reducing the severity of symptoms and integrin-related disorder-related effects, reducing the severity of an acute episode, reducing the number of symptoms and integrin-related disorder-related effects, reducing the latency of symptoms and integrin-related disorder-related effects, an amelioration of symptoms and integrin-related disorder-related effects, reducing secondary symptoms, reducing secondary infections, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and an increased survival time of the affected host animal, following administration of the agent/composition. A prophylactic effect can comprise a complete or partial avoidance/inhibition or a delay of integrin-related disorder development/progression, and an increased survival time of the affected host animal, following administration of the peptidomimetic compounds described herein.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). Metastasis can be determined by staging tests and tests for calcium level and other enzymes to determine the extent of metastasis. CT scans can also be done to look for spread to regions outside of the tumor or cancer.

The present disclosure also provides peptidomimetic compounds comprising the amino acid sequence NEDIDQMFSTLLGEMDLLTQS (SEQ ID NO:1) having from 0 to 5 conservative amino acid substitutions, modifications, deletions, or any combination thereof, or a pharmaceutically acceptable salt thereof, for use in treating a subject having an integrin-related disorder, wherein the compound comprises at least one structural stabilization moiety. The present disclosure also provides peptidomimetic compounds comprising the amino acid sequence NEDIDQMFSTLLGEMDLLTQS (SEQ ID NO:1) having from 0 to 5 conservative amino acid substitutions, modifications, deletions, or any combination thereof, or a pharmaceutically acceptable salt thereof, for use in the preparation of a medicament for treating a subject having an integrin-related disorder, wherein the compound comprises at least one structural stabilization moiety. Any of the peptidomimetic compounds described herein can be used.

In some embodiments, the at least one structural stabilization moiety links two nonadjacent amino acid residues within SEQ ID NO: 1. In some embodiments, the at least one structural stabilization moiety links any two non-adjacent amino acids within positions 10 to 21 of SEQ ID NO:1. In some embodiments, the at least one structural stabilization moiety links any two non-adjacent amino acids within positions 1 to 9 of SEQ ID NO:1. In some embodiments, the at least one structural stabilization moiety links any amino acid except 14, F8, L11, and L18 to another non-adjacent amino acid within SEQ ID NO:1. In some embodiments, the two nonadjacent amino acid residues within SEQ ID NO:1 are 2 to 7 amino acids apart. In some embodiments, the two nonadjacent amino acid residues within SEQ ID NO:1 are 2 amino acids apart. In some embodiments, the two nonadjacent amino acid residues within SEQ ID NO:1 are 3 amino acids apart. In some embodiments, the two nonadjacent amino acid residues within SEQ ID NO:1 are 4 amino acids apart. In some embodiments, the two nonadjacent amino acid residues within SEQ ID NO:1 are 5 amino acids apart. In some embodiments, the two nonadjacent amino acid residues within SEQ ID NO:1 are 6 amino acids apart. In some embodiments, the two nonadjacent amino acid residues within SEQ ID NO:1 are 7 amino acids apart. In some embodiments, the at least one structural stabilization moiety links the amino acid at position 15 to the amino acid at position 19 of SEQ ID NO:1. Any of the structural stabilization moiety links described herein can be used.

In some embodiments, the structural stabilization moiety is a hydrocarbon having from about 5 to about 20 carbons. In some embodiments, the hydrocarbon comprises a substituted or unsubstituted alkane comprising from about 5 to about 20 carbons, a substituted or unsubstituted alkene comprising from about 5 to about 20 carbons, or a substituted or unsubstituted alkyne comprising from about 5 to about 20 carbons. In some embodiments, the structural stabilization moiety is a hydrocarbon having from about 8 to about 12 carbons. In some embodiments, the hydrocarbon comprises a substituted or unsubstituted alkane comprising from about 8 to about 12 carbons, a substituted or unsubstituted alkene comprising from about 8 to about 12 carbons, or a substituted or unsubstituted alkyne comprising from about 8 to about 12 carbons. In some embodiments, the hydrocarbon comprises a substituted or unsubstituted alkane comprising about 8 carbons, a substituted or unsubstituted alkene comprising about 8 carbons, or a substituted or unsubstituted alkyne comprising about 8 carbons. In some embodiments, the structural stabilization moiety comprises a ring closing metathesis moiety, a copper catalyzed azide alkyne cycloaddition moiety, a lactamization reaction moiety, a cysteine-xylene stapling moiety, a cysteine-perfluorobenzene stapling moiety, a hiol-yne/-ene click chemistry group, a selenocysteine stapling moiety, a tryptophan condensation moiety, a C—H activation moiety, or a 1,3-dipolar cycloaddition stapling moiety. Any of the structural stabilization moieties described herein can be used.

In some embodiments, the peptidomimetic compound is fused to a heterologous molecule. In some embodiments, the heterologous molecule comprises an immunoglobulin Fc domain, a peptide purification tag, a fluorescent protein, or a transduction domain. In some embodiments, the peptidomimetic compound is linked to a label. In some embodiments, the label is a fluorescent label or a radiolabel. In some embodiments, the label comprises polyethylene glycol, polysialic acid, or glycolic acid. Any of the heterologous molecule described herein can be used.

In some embodiments, the integrin-related disorder is a thrombotic disorder, a cardiovascular disease, an autoimmune disease, or a cancer. In some embodiments, the thrombotic disorder is angina or restenosis. In some embodiments, the cardiovascular disease is stroke or acute coronary artery disease. In some embodiments, the autoimmune disease is asthma, psoriasis, multiple sclerosis, ulcerative colitis, rheumatoid arthritis, or Crohn's disease. In some embodiments, the cancer is renal cell carcinoma, metastatic melanoma, or pancreatic cancer. In some embodiments, the integrin-related disorder is also a talin-related disorder. Any of the integrin-related disorders described herein can be used.

The present disclosure provides methods of enhancing the permeability of a plasma membrane, the methods comprising contacting the plasma membrane with a peptidomimetic compound comprising the amino acid sequence NEDIDQMFSTLLGEMDLLTQS (SEQ ID NO:1) having from 0 to 5 conservative amino acid substitutions, or a pharmaceutically acceptable salt thereof, wherein the compound comprises at least one structural stabilization moiety. In some embodiments, the plasma membrane is disposed on a cell or platelet. In some embodiments, the cell comprises a lymphocyte. In some embodiments, the cell is in vivo. In some embodiments, the cell is in vitro.

The present disclosure provides peptidomimetic compounds comprising the amino acid sequence NEDIDQMFSTLLGEMDLLTQS (SEQ ID NO:1) having from 0 to 5 conservative amino acid substitutions, or a pharmaceutically acceptable salt thereof, for use in enhancing the permeability of a plasma membrane, wherein the compound comprises at least one structural stabilization moiety. In some embodiments, the plasma membrane is disposed on a cell or platelet. In some embodiments, the cell comprises a lymphocyte. In some embodiments, the cell is in vivo. In some embodiments, the cell is in vitro.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Synthesis of Stapled Peptides

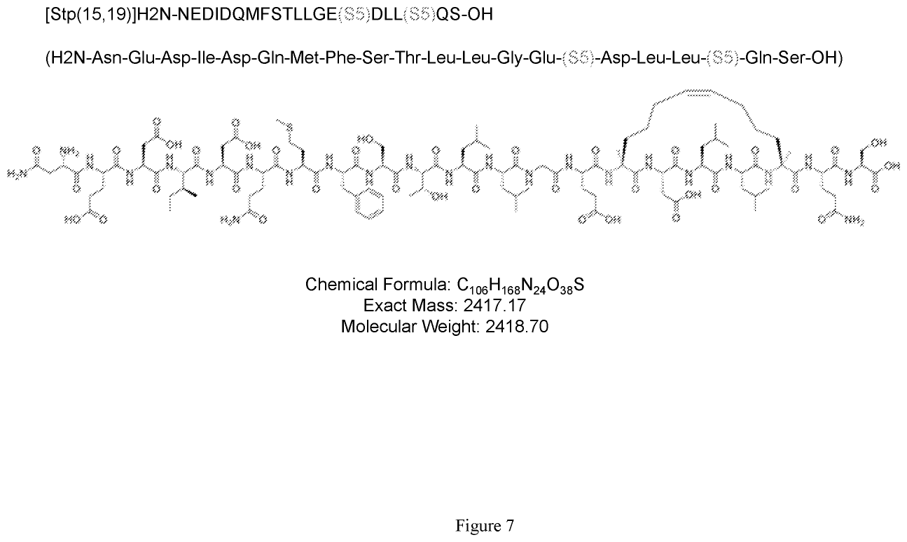

A stapled peptide with the sequence of NEDIDQMFSTLLGEMDLLTQS (SEQ ID NO:1) was synthesized by connecting the side chains of M15 and T19 using an 8-hydrocarbon link. The formula of (Stp(15,19))H₂N-NEDIDQMFSTLLGE(S5)DLL(S5)QS-OH (H₂N-Asn-Glu-Asp-Ile-Asp-Gln-Met-Phe-Ser-Thr-Leu-Leu-Gly-Glu-(S5)-Asp-Leu-Leu-(S5)-Gln-Ser-OH) is shown in FIG. 7.

### Example 2: X-ray Crystallography

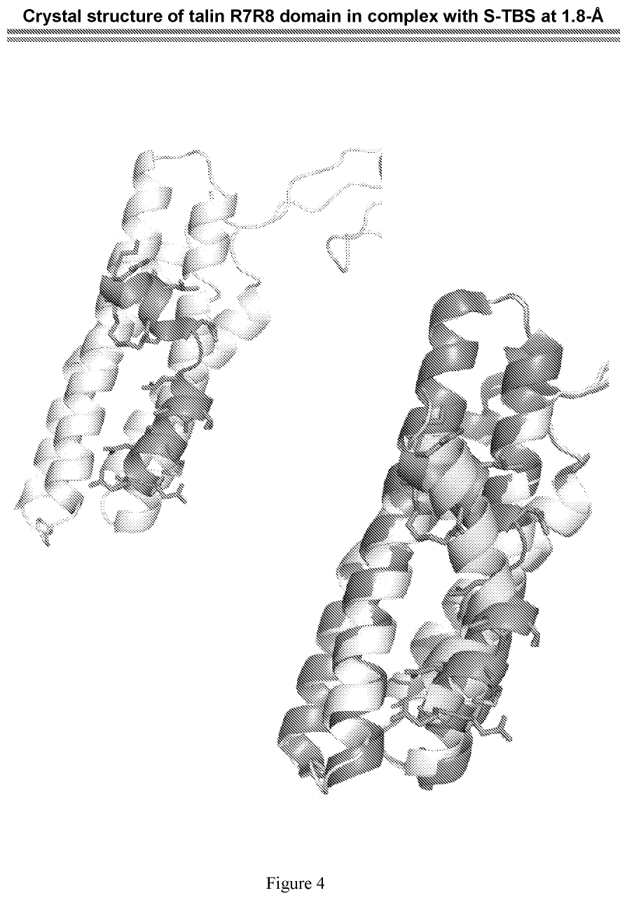

Talin R7R8 protein was purified as described previously (Chang et al., Structure, 2014, 22, 1810⁻¹⁸²⁰). Crystals of R7R8 in complex with the stapled-TBS peptide were obtained in a reservoir solution (100 mM NaCl, 20% (w/v) polyethylene glycol 3350 and 20% (v/v) ethylene glycol) from microseeding using R7R8/TBS crystals (Chang et al., Structure, 2014, 22, 1810-1820). The crystals were transferred to a cryo solution containing the reservoir solution and 25% ethylene glycol, and then flash-frozen in liquid nitrogen prior to the diffraction experiments. Diffraction data were collected at MacCHESS synchrotron. Structure of R7R8 in complex with the stapled-TBS peptide was determined by molecular replacement using the R7R8/TBS (PDB code: 4W8P) structure as the search model (see, FIG. 4).

### Example 3: Fluorescence-Activated Cell Sorting

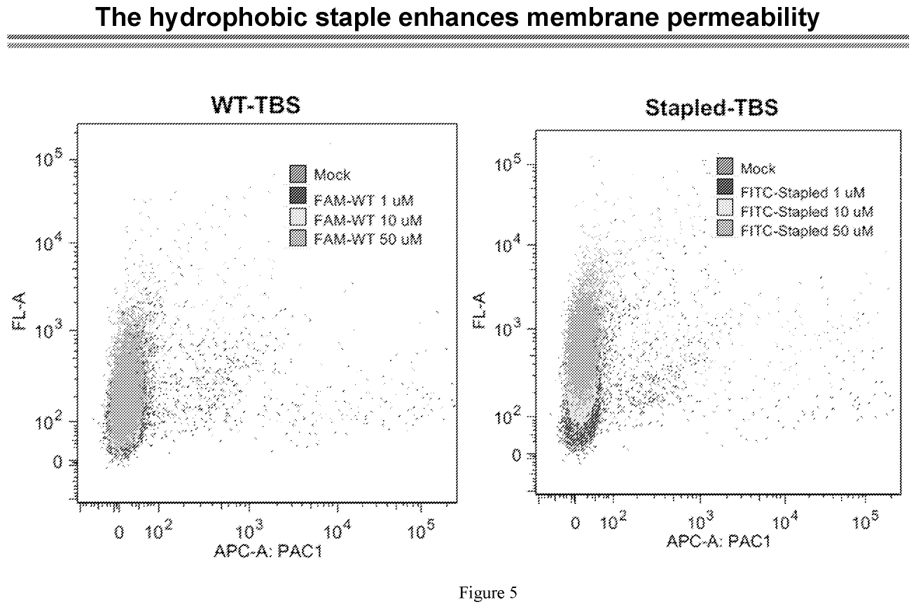

Assessing membrane permeability: CHO-A5 cells were treated with FAM-labelled WT-TBS (1, 10, and 50 μM), or with FITC-labelled stapled-TBS (1, 10, and 50 μM), and incubated for 30 minutes at 37° C. The cells were washed twice with phosphate-buffered saline (PBS), harvested in trypsin-EDTA, and centrifuged at 2,000 rpm for 5 minutes. The cells were resuspended in PBS and quantified with an LSR flow cytometer using 30,000 cells per measurement. The results are shown in FIG. 5.

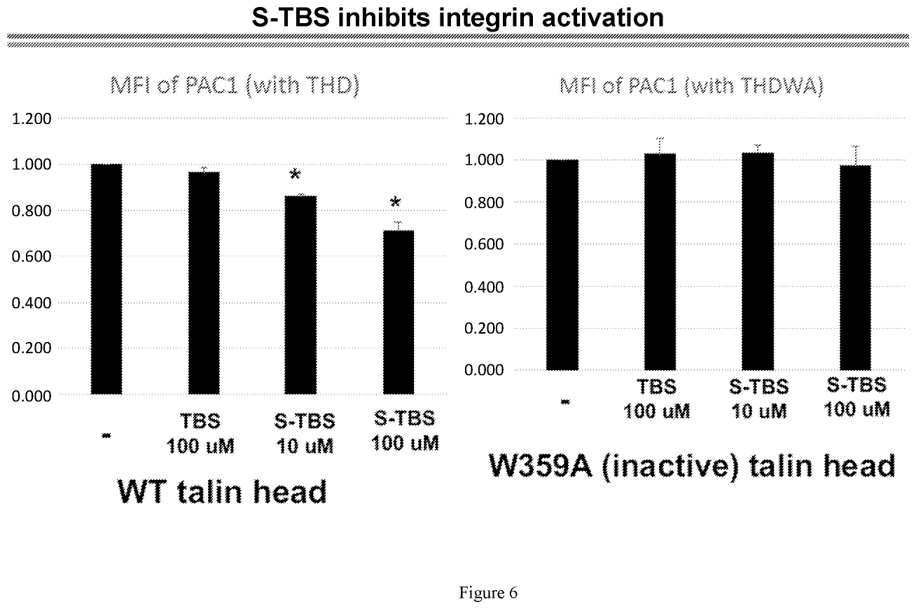

Assessing integrin activation: CHO-A5 cells were transfected with GFP-tagged talin-head domain (residues 1-430, WT or W359A) and incubated for 24 hours at 37° C. The cells were subsequently treated with WT-TBS at 100 μM or Staple-TBS (at 10 and 100 μM) for 30 minutes at 37° C. and washed twice with PBS, harvested in trypsin-EDTA, and centrifuged at 2,000 rpm for 5 minutes. The cells were incubated in Tyrode's buffer (136.9 mM NaCl, 10 mM HEPES, 5.5 mM glucose, 11.9 mM NaHCO₃, 2.7 mM KCl, 0.5 mM CaCl₂), 1.5 mM MgCl₂, and 0.4 mM NaH₂PO₄, pH 7.4) containing PAC1 antibody for 1 hour at 30° C. and then stained with Alexa 647 (goat anti-mouse IgM) for 30 minutes at RT. The cells were quantified with an LSR flow cytometer using 30,000 cells per measurement. The results are shown in FIG. 6.

### Example 4: Crystal Structure of S-TBS in Complex with Talin R7R8 Domains

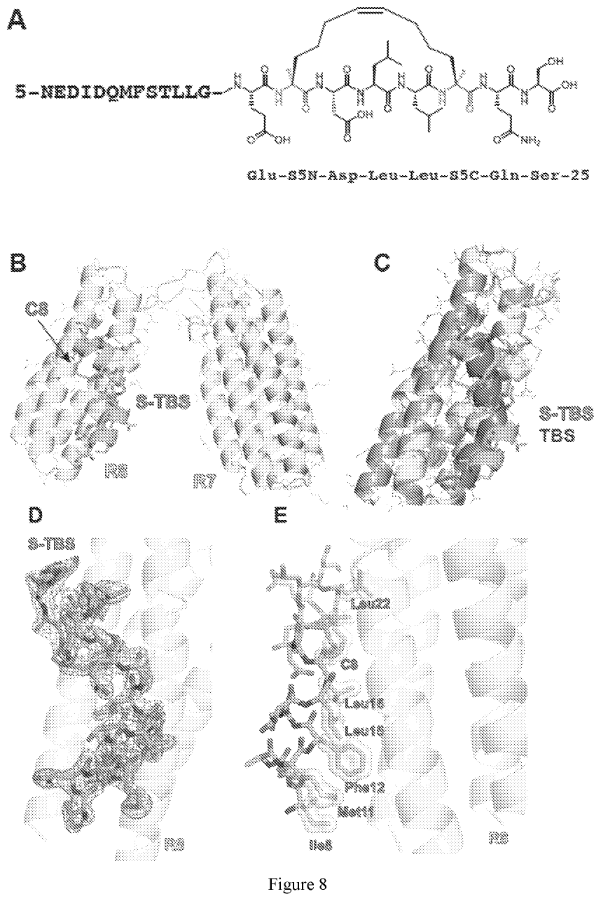

The TBS fragment binds to talin rod and talin head in a helical configuration, which is not stable in the unbound TBS peptide (see, Chang et al., 2014). To rigidify and pre-organize the scaffold of the TBS peptide to mimic the bound state, a C8 hydrocarbon staple was designed that connects two residues that are one helix apart (residues 19 and 23, FIG. 8 (Panel A)). The stapled peptide (S-TBS) possessing residues 5-25 of the TBS region in RIAM were synthesized and co-crystalized with recombinantly expressed talin R7R8 domains. Crystal structure of S-TBS in complex with a talin rod double-domain R7R8 was determined at 1.85-Å resolution:

TABLE 1
Crystallographic data collection and refinement statistics

| Data collection | | |
|---|---|---|
| Light Source | NSLS-II AMX | |
| Wavelength (Å) | 0.97918 | |
| Space group | P21212 | |
| Cell dimensions | | |
| a, b. c (Å) | 60.86, 104.33, 48.23 | |
| α, β, γ (°) | 90, 90, 90 | |
| Resolution (Å) | 104.33~1.85 | (1.88~1.85) |
| Rmerge (%) | 3.3 | (154.6) |
| I/σ | 22.6 | (1.0) |
| CC(½) | 99.9 | (47.5) |
| Completeness (%) | 99.4 | (91.9) |
| No. reflections | 27043 | (1512) |
| Multiplicity | 6.5 | (6.1) |

| | |
|---|---|
| Refinement | |
| Resolution (Å) | 52.57~1.85 |
| Rwork/Rfree (%) | 21.54/25.38 |
| No. atoms | |
| Protein | 2172 |
| Ligand/ion | 152 |
| Water | 144 |
| Wilson B factor (Å²) | |
| Protein | 60.6 |
| peptide | 87.44 |
| R.m.s deviations | |
| Bond lengths (Å) | 0.010 |
| Bond angles (°) | 1.343 |

The structure reveals that S-TBS binds to the R8 domain of talin rod and the binding site largely overlaps with that of the TBS peptide (FIG. 8, Panels B and C). Residues 8-25, including the two residues that are linked by the C8 staple, are well resolved (FIG. 8, Panel D). Interestingly, although bound S-TBS also exhibits a kinked-helical configuration, the helix “turns” at residue Leu15 instead of Ser13 as seen in the bound TBS. This change allows the side chain of Leu15 to make hydrophobic contacts with the β-carbons of Thr1496 and Ala1499. Indeed, the S-TBS peptide contacts the R8 domain via an interface that only contains hydrophobic residues, including Ile8, Met11, Phe12, Leu15, Leu16, Leu22, and the C8 staple that links residues 19 and 23 (FIG. 8, Panel E). The crystal structure suggests that the binding of S-TBS to the R8 domain is dominated by hydrophobic interactions. Moreover, it also indicates that the S-TBS peptide sterically competes against RIAM for talin rod binding.

### Example 5: S-TBS Binds to Talin Head Domain and Talin Rod R7R8 Much More Strongly than TBS

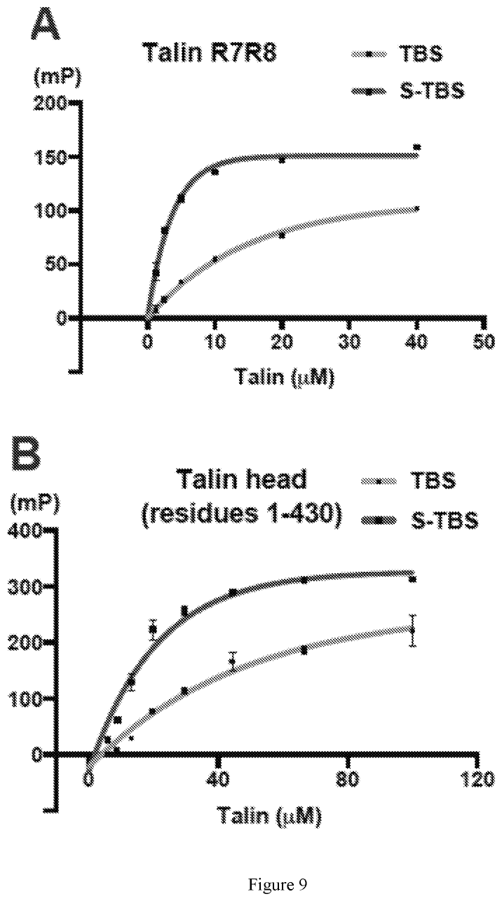

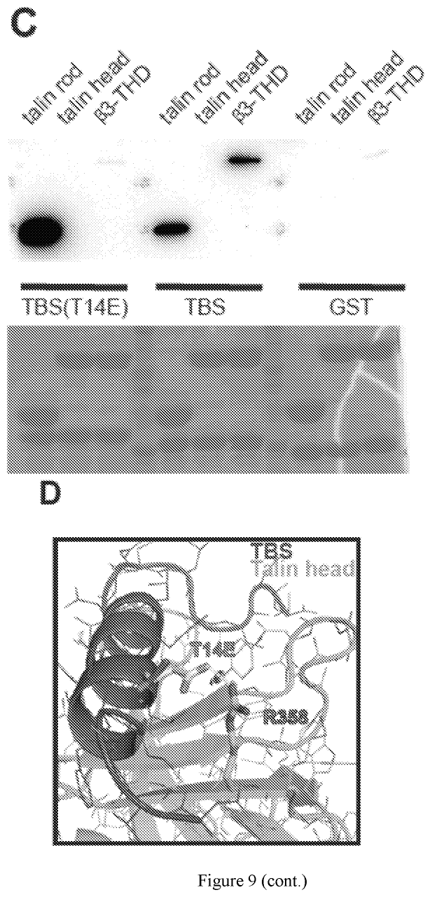

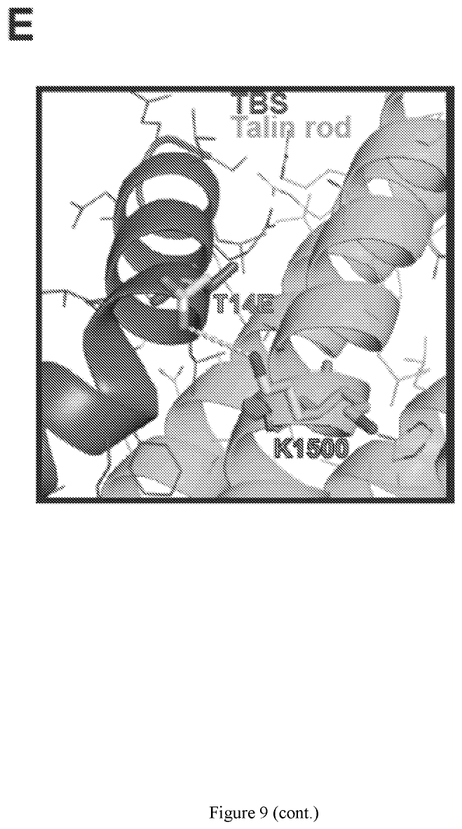

To assess the binding affinities of S-TBS with talin, the K_{d} of TBS and S-TBS peptides with talin rod R7R8 and talin head (residues 1-430) were measured using fluorophore-labeled peptides and recombinantly expressed, purified talin proteins by FP assays. The K_{d} values of S-TBS with R7R8 or with talin head are 3.0 μM and 30.3 μM, respectively (FIG. 9, Panels A and B, Table 2). These results are consistent with the low micromolar binding with R7R8 and mid-micromolar binding with talin head that were estimated in previous studies (see, Chang et al. 2014). Importantly, the binding of S-TBS is significantly stronger than that of TBS (6-fold stronger in binding to R7R8; 3.5-fold stronger in binding to talin head). This gain of affinity may be attributed to the C8 staple as it stabilizes the helical structure of TBS, and the enhanced hydrophobic interaction between S-TBS and talin rod. The enhanced binding of S-TBS to talin further validates the design strategy of using stapled peptides as a potent competitive inhibitor.

TABLE 2

| K_{d} (μM) | S-TBS | TBS |
|---|---|---|
| Talin rod (R7R8) | 3.0 ± 0.3 | 17.7 ± 1.3 |
| Talin head domain | 30.3 ± 4.8 | 116 ± 15.5 |
| Talin head/β3 | | |

### Example 6: S-TBS Competes with Integrin for Binding with the Talin Head Domain

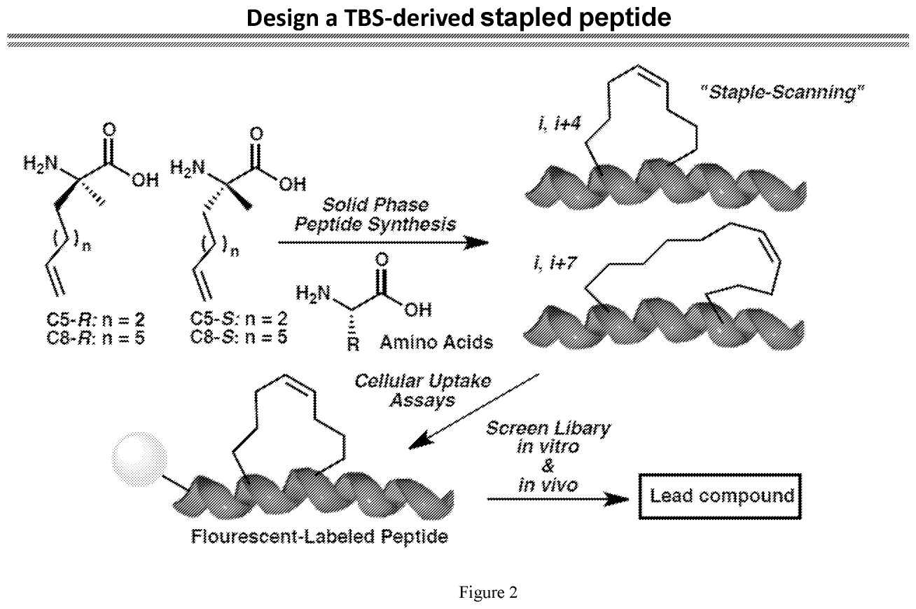

The interaction of the TBS segment of RIAM with the talin head domain is compatible with the integrin-β:talin interaction (see, Yang et al., Nat. Commun., 2014, 5, 5880). To test this proposed synergistic interaction, a chimeric protein was constructed by fusing a integrin 03 segment to talin head domain (β3-THD) and assessed its interaction with TBS. The GST-pulldown data indicate that TBS interacts with β3-THD much more strongly than with THD alone (FIG. 9, Panel C). This result strongly supports the synergistic interaction of integrin and talin by TBS. To further validate this result, a mutation in TBS that is predicted to compete against integrin-β for talin binding was generated. The T14E mutation in TBS is expected to generate anew salt-bridge interaction with talin through the Arg358 residue, which is essential for the interaction of talin and integrin-β3 (FIG. 2, Panel D; see Barsukov et al., J. Biol. Chem., 2003, 278, 31202-09). Although the pulldown assay was still unable to detect the interaction of TBS-T14E and talin head domain, as the synergistic model predicts, the T14E mutation significantly diminishes the interaction of TBS with β3-THD (FIG. 9, Panel C. Moreover, the T14E mutation may also generate a new salt-bridge interaction with talin R8 domain through Lys1500 (FIG. 9, Panel E). Indeed, TBS-T14E exhibits a stronger interaction with talin R7R8 domains (FIG. 9, Panel C). The binding affinities of S-TBS and β-THD by FP assays was determined. (FIG. 9, Panel F).

### Example 7: S-TBS Exhibits Much Greater Membrane Permeability than TBS

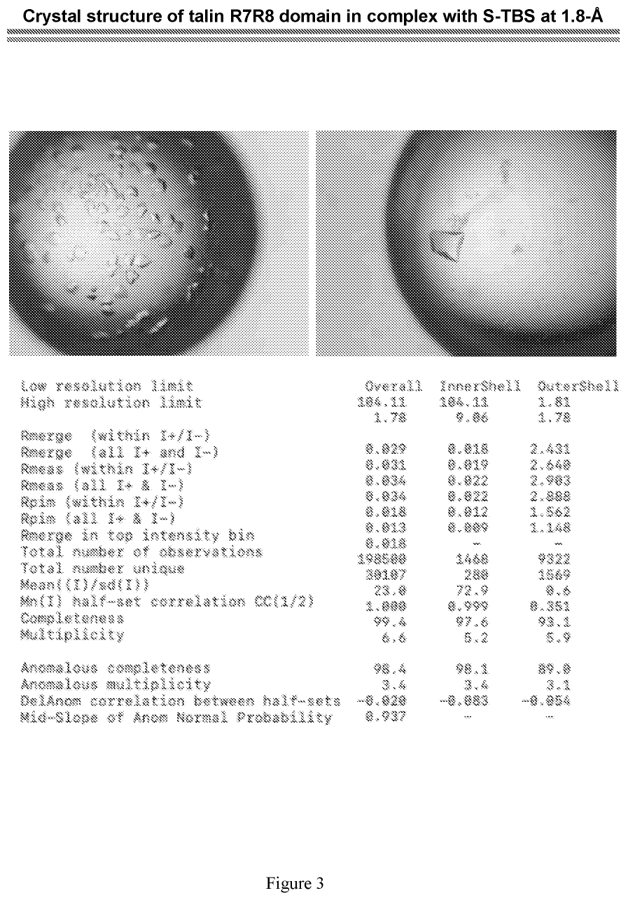

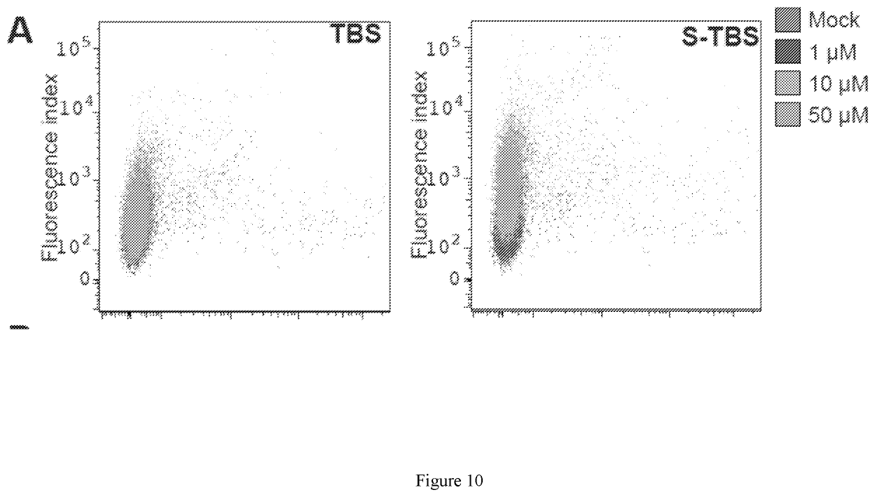

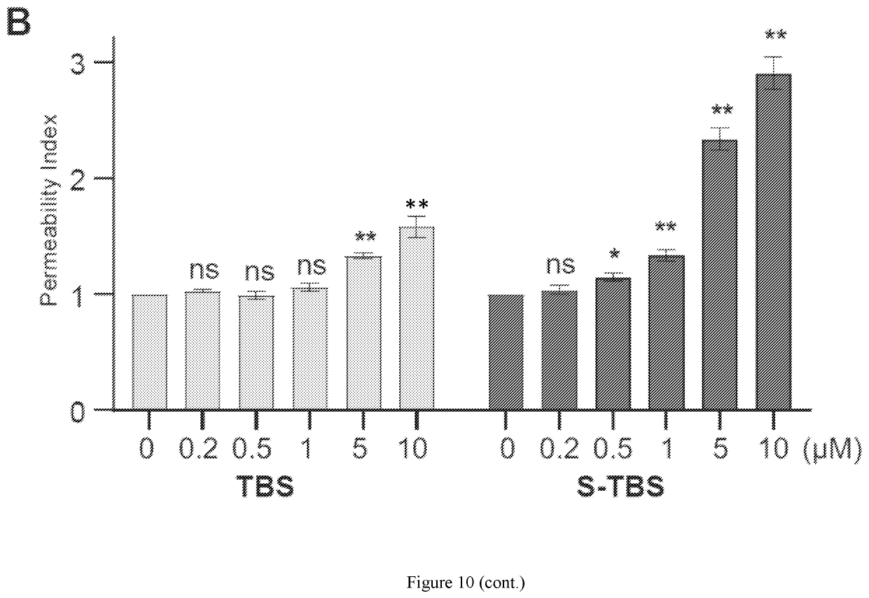

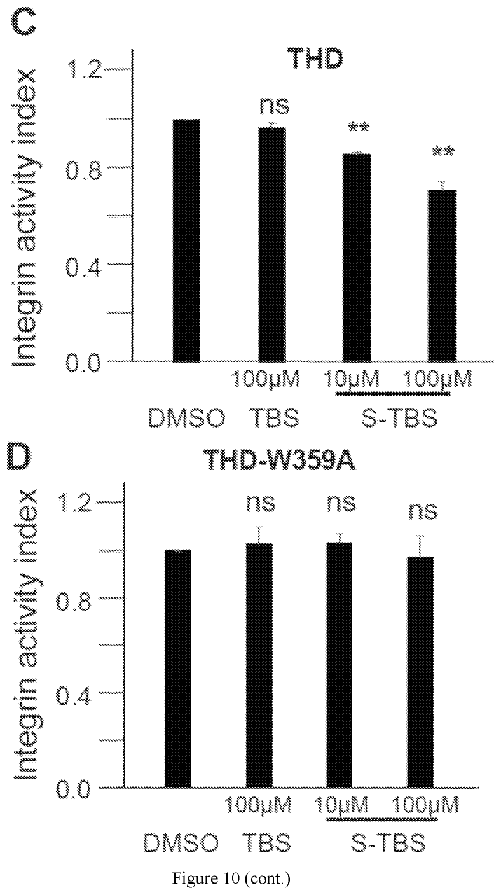

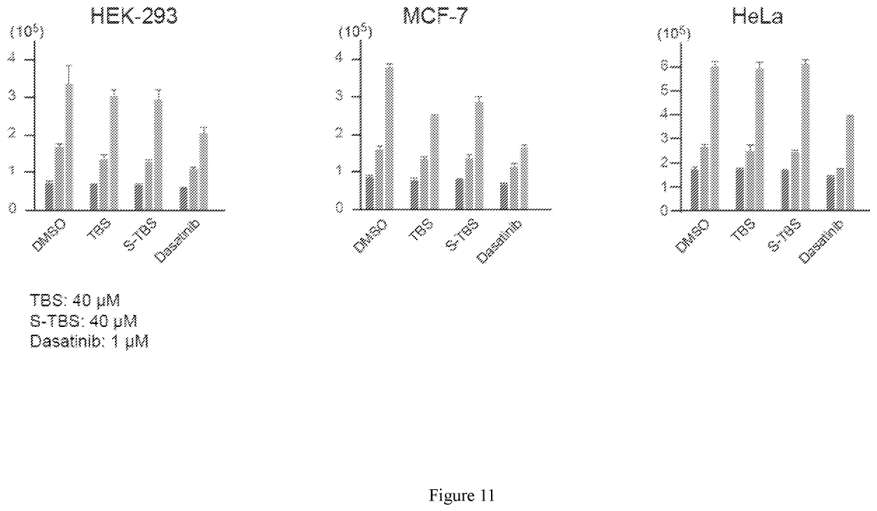

A common challenge for peptidomimetic drugs that target intracellular proteins is the poor cell membrane permeability. To assess the effect of the C8 staple on cell permeability, FITC-labeled S-TBS were incubated with CHO cells at various concentration and examined S-TBS uptake by flow cytometry. S-TBS treated cells exhibited significantly stronger signal in all testing concentrations (1, 10, and 50 μM) compared with the fluorophore-labelled TBS peptide (FIG. 10, Panel A)). The assay was then performed using lower doses of peptides and quantified the signals for statistical analyses. S-TBS exhibits significant cell uptake at 0.5 μM. In contrast, TBS requires a concentration of at least 5 μM for cell uptake (FIG. 3, Panel B). This result unexpectedly indicates that the hydrophobic C8 staple significantly improves the membrane permeability of S-TBS, thus overcoming a major obstacle for peptidomimetic drug design.

### Example 8: S-TBS Inhibits Talin-Induced Integrin Activation

The effect of S-TBS on talin-induced activation of integrin α_{IIb}β₃ was examined. CHO-A5 cells that stably express integrin α_{IIb}β₃ were transfected with a green fluorescent protein-tagged talin head domain, treated with S-TBS or TBS, and then subjected flow cytometry analysis for integrin α_{IIb}β₃ activity. S-TBS significantly suppresses integrin α_{IIb}β₃ activity in a dose-dependent manner, whereas the TBS peptide exhibits no inhibitory effect at 100 μM concentration (FIG. 10, Panel C). To verify that the inhibitory effect of S-TBS is talin specific, S-TBS was examined for its effect using CHO-A5 cells transfected with an inactive talin head mutation (THD-W359A). No inhibitory effect was observed in cells treated with both TBS or STBS peptides (FIG. 10, Panel D). This result confirms that S-TBS inhibits integrin activation in a talin-specific manner. The inhibitory effect of S-TBS is consistent with its competitive binding to talin head against integrin 03.

### Example 9: S-TBS Exhibits Minimal Cell Toxicity

The cell toxicity of the peptides was then examined. HEK, MCF-7, and HeLa were used. Cells were treated with S-TBS 40 μm for one hour. The S-TBS was then washed away and the viability measured at 24 and 48 hours. No impact on HEK, MCF-7, and HeLa cell growth was observed. Additionally, no impact on CHO cell or Jurkat T-cell growth suggesting the stapled peptide has minimal cell toxicity.

### Materials and Methods for Examples 4 to 9

Plasmid construction, protein purification, and GST pull-down: Talin R7R8, talin head domain, β3-THD chimera, and GST-TBS were expressed and purified as described previously described (see, Zhang et al., Proc. Natl. Acad. Sci. U.S.A., 2020, 117, 32402-12; and Chang et al., 2014). Point mutations were constructed using a site-directed mutagenesis method. For in vitro pull-down assays, purified GST-TBS, wild type or T14E, were immobilized on glutathione agarose beads and then incubated with purified His-tagged talin proteins in reaction buffer (20 mM Tris-HCl pH 7.5, 100 mM NaCl, and 2 mM DTT) to a total volume of 250 μl on a rotator for one hour at 4° C. The bound proteins were washed three times in 500 μl of the lysis buffer and were eluted using an elution buffer (20 mM Tris-HCl pH 7.5, 100 mM NaCl, 2 mM DTT, and 10 mM reduced glutathione) at 4° C. The proteins were resolved by SDS-PAGE and detected by Coomassie staining or Western blotting. The Immobilon-P transfer membranes (EMD Millipore) were blocked with TBST buffer (20 mM Tris-HCl pH 7.5, 150 mM NaCl, 0.1% (v/v) Tween 20) containing 5% (w/v) BSA for one hour and then incubated with anti-His (Sigma) or anti-GFP antibody (Clontech) for 1 hour at room temperature followed by a second incubation with HRP-conjugated secondary antibody (Santa Cruz Biotechnology). The blots were visualized with the SuperSignal West Pico Chemiluminescent Substrate (Thermo Scientific) and detected using the FluorChem E imager (ProteinSimple).

Crystallization and structure determination method: The talin rod domains R7R8 was purified as described in previous study (Chang et al., 2014). The S-TBS peptide (New England Peptide), possessing mouse RIAM residues 5-25 with a C8 linker between residues 19 and 23, was solubilized in 20 mM Tris pH8.0, 100 mM NaCl with 50% DMSO. Talin R7R8 (35 mg/mL) was incubated with the S-TBS on ice at a 1:3 molar ratio prior to the crystallization setup. STBS:R7R8 complex was crystallized using the hanging-drop vapor diffusion method at room temperature in 100 mM NaCl, 20% (w/v) polyethylene glycol 3350 and 20% (v/v) ethylene glycol. X-ray diffraction data was collected at APS NECAT 24-ID-E beamline. The collected datasets were indexed, integrated and scaled with XDS (see, Kabsch, Acta Crystallogr. D Biol. Crystallogr. 2010, 66, 125-32). Data collection and refinement statistics are listed in Table 1. The crystal structure was determined by molecular replacement using R7R8 structure (pdb: 4w8p) as a search model. This model was iteratively built in COOT (40). and refined in PHENIX and REFMAC (see, Vagin et al., Acta Crystallogr. D Biol. Crystallogr., 2004, 60, 2184-95). To build the model of the stapled peptide, Met19 and Thr23 was replaced by irregular amino acid MK8 and the side chain was linked.

Fluorescent Polarization: Mixtures were prepared of purified talin proteins at a series concentration (R7R8: 0.2 μM to 20 μM; THD/b3-THD: 5 μM to 100 μM), or buffer only, with 20 nM FITC-labeled S-TBS (New England Peptide) in a buffer containing 20 mM HEPES, pH 7.5, 100 mM NaCl, and 2 mM DTT. After five minutes of incubation, 20 μl of the reaction mix was added to individual wells of a 384-well assay plate (Corning) and then measured fluorescence polarization at room temperature using a plate reader. A 535/25 nm filter was used as an excitation filter, and a pair of 590/20 nm filters as emission polarization filters. FP signals were normalized against buffer-only background and then fit to a single-site (saturating) binding model using SigmaPlot (Systat Software).

Cell permeability assay: CHO-A5 cells were plated in 6-well dishes, and then treated with fluorophore labeled TBS or S-TBS peptides at indicated concentrations for 30 minutes at 37° C. The cells were then washed twice with phosphate-buffered saline (PBS), detached by adding a mixture of trypsin and ethylene glycol-bis(O-aminoethyl ether)—N,N,N′,N′-tetraacetic acid (“trypsin-EDTA”) and harvested by spinning at 2,000 rpm for 5 minutes. The cells were resuspended in PBS and quantified with an LSR flow cytometer (525/50 filter, 488 nm laser) using 30,000 cells per measurement.

Integrin activation assays: CHO-A5 cells were transfected with talin-head domain, wild-type or W359A, and incubated for 24 hours at 37° C. The cells were split to achieve equal expression level of talin head, and subsequently treated with TBS peptide or S-TBS for 30 minutes at 37° C. and washed twice with PBS buffer. The cells were then detached by adding trypsin-EDTA, and harvested by spinning at 2,000 rpm for 5 minutes. The cells were incubated in Tyrode's buffer (136.9 mM NaCl, 10 mM HEPES, 5.5 mM Glucose, 11.9 mM NaHCO₃, 2.7 mM KCl, 0.5 mM CaCl₂), 1.5 mM MgCl₂, 0.4 mM NaH₂PO₄, pH 7.4) containing the PAC1 antibody (Becton, Dickenson & Co.) for one hour at 30° C. and then stained with Alexa 647 goat anti-mouse IgM (Invitrogen) for 30 minutes at room temperature. The cells were quantified with a Beckton, Dickenson & Co. Biosciences LSR flow cytometer (660/20 filter, 640 nm laser) using 30,000 cells per measurement.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A peptidomimetic compound comprising the amino acid sequence NEDIDQMFSTL LGEMDLLTQS (SEQ ID NO:1) having from 0 to 5 conservative amino acid substitutions, or a pharmaceutically acceptable salt thereof, wherein the compound comprises at least one structural stabilization moiety.

2. The peptidomimetic compound of claim 1, wherein the at least one structural stabilization moiety links two nonadjacent amino acid residues within SEQ ID NO:1.

3. The peptidomimetic compound of claim 1 or claim 2, wherein the at least one structural stabilization moiety links any two non-adjacent amino acids within positions 10 to 21 of SEQ ID NO:1.

4. The peptidomimetic compound of claim 1 or claim 2, wherein the at least one structural stabilization moiety links any two non-adjacent amino acids within positions 1 to 9 of SEQ ID NO:1.

5. The peptidomimetic compound of any one of claims 1 to 4, wherein the at least one structural stabilization moiety links any amino acid except 14, F8, L11, and L18 to another non-adjacent amino acid within SEQ ID NO:1.

6. The peptidomimetic compound of any one of claims 2 to 5, wherein the two nonadjacent amino acid residues within SEQ ID NO:1 are 2 to 7 amino acids apart.

7. The peptidomimetic compound of any one of claims 2 to 5, wherein the two nonadjacent amino acid residues within SEQ ID NO:1 are 2 amino acids apart.

8. The peptidomimetic compound of any one of claims 2 to 5, wherein the two nonadjacent amino acid residues within SEQ ID NO:1 are 3 amino acids apart.

9. The peptidomimetic compound of any one of claims 2 to 5, wherein the two nonadjacent amino acid residues within SEQ ID NO:1 are 4 amino acids apart.

10. The peptidomimetic compound of any one of claims 2 to 5, wherein the two nonadjacent amino acid residues within SEQ ID NO:1 are 5 amino acids apart.

11. The peptidomimetic compound of any one of claims 2 to 5, wherein the two nonadjacent amino acid residues within SEQ ID NO:1 are 6 amino acids apart.

12. The peptidomimetic compound of any one of claims 2 to 5, wherein the two nonadjacent amino acid residues within SEQ ID NO:1 are 7 amino acids apart.

13. The peptidomimetic compound of claim 1, wherein the at least one structural stabilization moiety links the amino acid at position 15 to the amino acid at position 19 of SEQ ID NO:1

14. The peptidomimetic compound of any one of claims 1 to 13, wherein the structural stabilization moiety is a hydrocarbon having from about 5 to about 20 carbons.

15. The peptidomimetic compound of claim 14, wherein the hydrocarbon comprises a substituted or unsubstituted alkane comprising from about 5 to about 20 carbons, a substituted or unsubstituted alkene comprising from about 5 to about 20 carbons, or a substituted or unsubstituted alkyne comprising from about 5 to about 20 carbons.

16. The peptidomimetic compound of any one of claims 1 to 13, wherein the structural stabilization moiety is a hydrocarbon having from about 8 to about 12 carbons.

17. The peptidomimetic compound of claim 16, wherein the hydrocarbon comprises a substituted or unsubstituted alkane comprising from about 8 to about 12 carbons, a substituted or unsubstituted alkene comprising from about 8 to about 12 carbons, or a substituted or unsubstituted alkyne comprising from about 8 to about 12 carbons.

18. The peptidomimetic compound of claim 16, wherein the hydrocarbon comprises a substituted or unsubstituted alkane comprising about 8 carbons, a substituted or unsubstituted alkene comprising about 8 carbons, or a substituted or unsubstituted alkyne comprising about 8 carbons.

19. The peptidomimetic compound of any one of claims 1 to 13, wherein the structural stabilization moiety comprises a ring closing metathesis moiety, a copper catalyzed azide alkyne cycloaddition moiety, a lactamization reaction moiety, a cysteine-xylene stapling moiety, a cysteine-perfluorobenzene stapling moiety, a hiol-yne/-ene click chemistry group, a selenocysteine stapling moiety, a tryptophan condensation moiety, a C—H activation moiety, or a 1,3-dipolar cycloaddition stapling moiety.

20. The peptidomimetic compound of any one of claims 1 to 19, wherein the peptidomimetic compound is fused to a heterologous molecule.

21. The peptidomimetic compound of claim 20, wherein the heterologous molecule comprises an immunoglobulin Fc domain, a peptide purification tag, a fluorescent protein, or a transduction domain.

22. The peptidomimetic compound of any one of claims 1 to 21, wherein the peptidomimetic compound is linked to a label.

23. The peptidomimetic compound of claim 22, wherein the label is a fluorescent label or a radiolabel.

24. The peptidomimetic compound of claim 22, wherein the label comprises polyethylene glycol, polysialic acid, or glycolic acid.

25. A composition comprising the peptidomimetic compound of any one of claims 1 to 24 and a carrier or excipient.

26. A method of producing the peptidomimetic compound of any one of claims 1 to 24, comprising:
culturing a host cell comprising a nucleic acid molecule encoding a peptide comprising the amino acid sequence NEDIDQMFSTLLGEMDLLTQS (SEQ ID NO:1) having from 0 to 5 conservative amino acid substitutions, whereby the host cell expresses the peptide;
recovering the expressed peptide from the host cell culture; and
linking two non-adjacent amino acids within SEQ ID NO:1 with at least one structural stabilization moiety.

27. The method of claim 26, the method further comprising conjugating the peptidomimetic compound to a heterologous molecule or a label.

28. The method according to claim 26 or claim 27, wherein the nucleic acid molecule is under the control of a heterologous promoter.

29. The method according to any one of claims 26 to 28, wherein the nucleic acid molecule is under the control of an inducible promoter.

30. A method of treating a subject having an integrin-related disorder, the method comprising administering a peptidomimetic compound comprising the amino acid sequence NEDIDQMFSTLLGEMDLLTQS (SEQ ID NO:1) having from 0 to 5 conservative amino acid substitutions, or a pharmaceutically acceptable salt thereof, wherein the compound comprises at least one structural stabilization moiety.

31. The method of claims 30 or 93, wherein the at least one structural stabilization moiety links two nonadjacent amino acid residues within SEQ ID NO:1.

32. The method of claim 30 or claim 31, wherein the at least one structural stabilization moiety links any two non-adjacent amino acids within positions 10 to 21 of SEQ ID NO:1.

33. The method of claim 30 or claim 31, wherein the at least one structural stabilization moiety links any two non-adjacent amino acids within positions 1 to 9 of SEQ ID NO:1.

34. The method of any one of claims 30 to 33, wherein the at least one structural stabilization moiety links any amino acid except 14, F8, L11, and L18 to another non-adjacent amino acid within SEQ ID NO:1.

35. The method of any one of claims 31 to 34, wherein the two nonadjacent amino acid residues within SEQ ID NO:1 are 2 to 7 amino acids apart.

36. The method of any one of claims 31 to 34, wherein the two nonadjacent amino acid residues within SEQ ID NO:1 are 2 amino acids apart.

37. The method of any one of claims 31 to 34, wherein the two nonadjacent amino acid residues within SEQ ID NO:1 are 3 amino acids apart.

38. The method of any one of claims 31 to 34, wherein the two nonadjacent amino acid residues within SEQ ID NO:1 are 4 amino acids apart.

39. The method of any one of claims 31 to 34 wherein the two nonadjacent amino acid residues within SEQ ID NO:1 are 5 amino acids apart.

40. The method of any one of claims 31 to 34, wherein the two nonadjacent amino acid residues within SEQ ID NO:1 are 6 amino acids apart.

41. The method of any one of claims 31 to 34, wherein the two nonadjacent amino acid residues within SEQ ID NO:1 are 7 amino acids apart.

42. The method of claim 30 or 93, wherein the at least one structural stabilization moiety links the amino acid at position 15 to the amino acid at position 19 of SEQ ID NO:1

43. The method of any one of claims 30 to 42, wherein the structural stabilization moiety is a hydrocarbon having from about 5 to about 20 carbons.

44. The method of claim 43, wherein the hydrocarbon comprises a substituted or unsubstituted alkane comprising from about 5 to about 20 carbons, a substituted or unsubstituted alkene comprising from about 5 to about 20 carbons, or a substituted or unsubstituted alkyne comprising from about 5 to about 20 carbons.

45. The method of any one of claims 30 to 42, wherein the structural stabilization moiety is a hydrocarbon having from about 8 to about 12 carbons.

46. The method of claim 45, wherein the hydrocarbon comprises a substituted or unsubstituted alkane comprising from about 8 to about 12 carbons, a substituted or unsubstituted alkene comprising from about 8 to about 12 carbons, or a substituted or unsubstituted alkyne comprising from about 8 to about 12 carbons.

47. The method of claim 45, wherein the hydrocarbon comprises a substituted or unsubstituted alkane comprising about 8 carbons, a substituted or unsubstituted alkene comprising about 8 carbons, or a substituted or unsubstituted alkyne comprising about 8 carbons.

48. The method of any one of claims 30 to 42, wherein the structural stabilization moiety comprises a ring closing metathesis moiety, a copper catalyzed azide alkyne cycloaddition moiety, a lactamization reaction moiety, a cysteine-xylene stapling moiety, a cysteine-perfluorobenzene stapling moiety, a hiol-yne/-ene click chemistry group, a selenocysteine stapling moiety, a tryptophan condensation moiety, a C—H activation moiety, or a 1,3-dipolar cycloaddition stapling moiety.

49. The method of any one of claims 30 to 48, wherein the peptidomimetic compound is fused to a heterologous molecule.

50. The method of claim 49, wherein the heterologous molecule comprises an immunoglobulin Fc domain, a peptide purification tag, a fluorescent protein, or a transduction domain.

51. The method of any one of claims 30 to 50, wherein the peptidomimetic compound is linked to a label.

52. The method of claim 51, wherein the label is a fluorescent label or a radiolabel.

53. The method of claim 51, wherein the label comprises polyethylene glycol, polysialic acid, or glycolic acid.

54. The method of any one of claims 30 to 53, wherein the integrin-related disorder is a thrombotic disorder, a cardiovascular disease, an autoimmune disease, or a cancer.

55. The method of claim 54, wherein the thrombotic disorder is angina or restenosis.

56. The method of claim 54, wherein the cardiovascular disease is stroke or acute coronary artery disease.

57. The method of claim 54, wherein the autoimmune disease is asthma, psoriasis, multiple sclerosis, ulcerative colitis, rheumatoid arthritis, or Crohn's disease.

58. The method of claim 54, wherein the cancer is renal cell carcinoma, metastatic melanoma, or pancreatic cancer.

59. A peptidomimetic compound comprising the amino acid sequence NEDIDQMFSTL LGEMDLLTQS (SEQ ID NO:1) having from 0 to 5 conservative amino acid substitutions, or a pharmaceutically acceptable salt thereof, for use in treating a subject having an integrin-related disorder, wherein the compound comprises at least one structural stabilization moiety.

60. The peptidomimetic compound of claims 59 or 99, wherein the at least one structural stabilization moiety links two nonadjacent amino acid residues within SEQ ID NO:1.

61. The peptidomimetic compound of claim 59 or claim 60, wherein the at least one structural stabilization moiety links any two non-adjacent amino acids within positions 10 to 21 of SEQ ID NO:1.

62. The peptidomimetic compound of claim 59 or claim 60, wherein the at least one structural stabilization moiety links any two non-adjacent amino acids within positions 1 to 9 of SEQ ID NO:1.

63. The peptidomimetic compound of any one of claims 59 to 62, wherein the at least one structural stabilization moiety links any amino acid except 14, F8, L11, and L18 to another non-adjacent amino acid within SEQ ID NO:1.

64. The peptidomimetic compound of any one of claims 60 to 63, wherein the two nonadjacent amino acid residues within SEQ ID NO:1 are 2 to 7 amino acids apart.

65. The peptidomimetic compound of any one of claims 60 to 63, wherein the two nonadjacent amino acid residues within SEQ ID NO:1 are 2 amino acids apart.

66. The peptidomimetic compound of any one of claims 58 to 63, wherein the two nonadjacent amino acid residues within SEQ ID NO:1 are 3 amino acids apart.

67. The peptidomimetic compound of any one of claims 58 to 63, wherein the two nonadjacent amino acid residues within SEQ ID NO:1 are 4 amino acids apart.

68. The peptidomimetic compound of any one of claims 58 to 63, wherein the two nonadjacent amino acid residues within SEQ ID NO:1 are 5 amino acids apart.

69. The peptidomimetic compound of any one of claims 58 to 63, wherein the two nonadjacent amino acid residues within SEQ ID NO:1 are 6 amino acids apart.

70. The peptidomimetic compound of any one of claims 58 to 63, wherein the two nonadjacent amino acid residues within SEQ ID NO:1 are 7 amino acids apart.

71. The peptidomimetic compound of claims 59 or 99, wherein the at least one structural stabilization moiety links the amino acid at position 15 to the amino acid at position 19 of SEQ ID NO:1.

72. The peptidomimetic compound of any one of claims 59 to 71, wherein the structural stabilization moiety is a hydrocarbon having from about 5 to about 20 carbons.

73. The peptidomimetic compound of claim 72, wherein the hydrocarbon comprises a substituted or unsubstituted alkane comprising from about 5 to about 20 carbons, a substituted or unsubstituted alkene comprising from about 5 to about 20 carbons, or a substituted or unsubstituted alkyne comprising from about 5 to about 20 carbons.

74. The peptidomimetic compound of any one of claims 59 to 71, wherein the structural stabilization moiety is a hydrocarbon having from about 8 to about 12 carbons.

75. The peptidomimetic compound of claim 74, wherein the hydrocarbon comprises a substituted or unsubstituted alkane comprising from about 8 to about 12 carbons, a substituted or unsubstituted alkene comprising from about 8 to about 12 carbons, or a substituted or unsubstituted alkyne comprising from about 8 to about 12 carbons.

76. The peptidomimetic compound of claim 74, wherein the hydrocarbon comprises a substituted or unsubstituted alkane comprising about 8 carbons, a substituted or unsubstituted alkene comprising about 8 carbons, or a substituted or unsubstituted alkyne comprising about 8 carbons.

77. The peptidomimetic compound of any one of claims 59 to 71, wherein the structural stabilization moiety comprises a ring closing metathesis moiety, a copper catalyzed azide alkyne cycloaddition moiety, a lactamization reaction moiety, a cysteine-xylene stapling moiety, a cysteine-perfluorobenzene stapling moiety, a hiol-yne/-ene click chemistry group, a selenocysteine stapling moiety, a tryptophan condensation moiety, a C—H activation moiety, or a 1,3-dipolar cycloaddition stapling moiety.

78. The peptidomimetic compound of any one of claims 59 to 77, wherein the peptidomimetic compound is fused to a heterologous molecule.

79. The peptidomimetic compound of claim 78, wherein the heterologous molecule comprises an immunoglobulin Fc domain, a peptide purification tag, a fluorescent protein, or a transduction domain.

80. The peptidomimetic compound of any one of claims 59 to 79, wherein the peptidomimetic compound is linked to a label.

81. The peptidomimetic compound of claim 80, wherein the label is a fluorescent label or a radiolabel.

82. The peptidomimetic compound of claim 80, wherein the label comprises polyethylene glycol, polysialic acid, or glycolic acid.

83. The peptidomimetic compound of any one of claims 59 to 82, wherein the integrin-related disorder is a thrombotic disorder, a cardiovascular disease, an autoimmune disease, or a cancer.

84. The peptidomimetic compound of claim 83, wherein the thrombotic disorder is angina or restenosis.

85. The peptidomimetic compound of claim 83, wherein the cardiovascular disease is stroke or acute coronary artery disease.

86. The peptidomimetic compound of claim 83, wherein the autoimmune disease is asthma, psoriasis, multiple sclerosis, ulcerative colitis, rheumatoid arthritis, or Crohn's disease.

87. The peptidomimetic compound of claim 83, wherein the cancer is renal cell carcinoma, metastatic melanoma, or pancreatic cancer.

88. A method of enhancing the permeability of a plasma membrane, the method comprising contacting the plasma membrane with a peptidomimetic compound comprising the amino acid sequence NEDIDQMFSTLLGEMDLLTQS (SEQ ID NO:1) having from 0 to 5 conservative amino acid substitutions, or a pharmaceutically acceptable salt thereof, wherein the compound comprises at least one structural stabilization moiety.

89. The method of claim 88, wherein the plasma membrane is a plasma membrane of a cell or platelet.

90. The method of claim 89, wherein the cell comprises a lymphocyte.

91. The method of claims 89 or 90, wherein the cell is in vivo.

92. The method of any of claims 89 to 91, wherein the cell is in vitro.

93. The method of any of claims 89 to 91, wherein the cell is disposed within a subject having an integrin-related disorder.

94. A peptidomimetic compound comprising the amino acid sequence NEDIDQMFSTL LGEMDLLTQS (SEQ ID NO:1) having from 0 to 5 conservative amino acid substitutions, or a pharmaceutically acceptable salt thereof, for use in enhancing the permeability of a plasma membrane, wherein the compound comprises at least one structural stabilization moiety.

95. The peptidomimetic compound of claim 94, wherein the plasma membrane is a plasma membrane of a cell or platelet.

96. The peptidomimetic compound of claim 95, wherein the cell comprises a lymphocyte.

97. The peptidomimetic compound of claims 95 or 96, wherein the cell is in vivo.

98. The peptidomimetic compound of claims 95 or 96, wherein the cell is in vitro.

99. The peptidomimetic compound of any of claims 95 to 96, wherein the peptidomimetic compound is also for use in a subject having an integrin-related disorder.
